(19) United States
(12) Patent Application Publication
(10) Pub. No.: US 2015/0037269 A1
(43) Pub. Date: Feb. 5, 2015

(54) TOPICAL COMPOSITIONS FOR TREATMENT OF EXCESSIVE SWEATING AND METHODS OF USE THEREOF

(71) Applicant: GlaxoSmithKline Intellectual Property Development Limited, Brentford (GB)

(72) Inventors: Teodora Pene DUMITRESCU, Research Triangle Park, NC (US); Elizabeth K. HUSSEY, Research Triangle Park, NC (US); Maria Graziella LARM, Rosanna (AU); Jon LENN, Research Triangle Park, NC (US); Leon LOUPENOK, Rowville (AU); Michael R. LUKE, Rowville (AU); Leandro L. SANTOS, Research Triangle Park, NC (US); Virginia D. SCHMITH, Research Triangle Park, NC (US)

(21) Appl. No.: 14/446,820

(22) Filed: Jul. 30, 2014

Related U.S. Application Data
(60) Provisional application No. 61/859,947, filed on Jul. 30, 2013.
(60) Provisional application No. 61/952,238, filed on Mar. 13, 2014.
(60) Provisional application No. 62/021,878, filed on Jul. 8, 2014.

(51) Int. Cl. A61K 8/49; A61Q 15/00

(52) U.S. Cl. CPC A61K 8/4926; A61Q 15/00; A61K 2800/10
USPC 424/65

(57) ABSTRACT

The present invention provides for 4-[hydroxy(diphenyl)methyl]-1-{2-[(phenylmethyl)oxy]ethyl}-1-azoniabicyclo[2.2.2]octane and a pharmaceutically acceptable anion thereof for use in the topical treatment or prophylaxis of excessive sweating, and compositions containing these ingredients.

### RELATED APPLICATIONS

This application claims the benefit of priority from U.S. Provisional Application No.'s 61/859,947, 61/952,238, and 62/021,878.

### FIELD OF THE DISCLOSURE

This disclosure relates to topical pharmaceutical compositions for use in the treatment of excessive sweating and methods of use thereof.

### BACKGROUND OF THE DISCLOSURE

Hyperhidrosis, a condition that affects approximately 3.0% of the US population, is defined as excessive sweating beyond what is physiologically required to maintain normal thermal regulation of the body [Strutton et al., J Am Acad Dermatol. 2004; 51:241-248]. Primary hyperhidrosis (excessive sweating without an alternative explanation) is focal (localized), and can affect the axilla (underarm), palms, soles of the feet, face, groin, trunk and thighs. Secondary hyperhidrosis can be either focal or generalized (entire body) and results from any number of medical conditions, including endocrine, metabolic, neurologic and cardiovascular disorders, and from medication use.

As noted, the most active regions of perspiration include the hands, feet, armpits and groin areas. Focal hyperhidrosis is when the excessive sweating is localized, e.g. it affects a specific area such as palmoplantar hyperhidrosis (also known as acrohyperhidrosis), which is the symptomatic sweating of primarily the hands (palms) and/or feet (soles). Generalized hyperhidrosis is the excessive sweating of the entire body.

Hyperhidrosis can have a debilitating effect on a patient's quality of life. Excessive sweating of the armpits, hands, feet or face can result in substantial impairment for the patient, including limitations in work, social interaction, physical activity and leisure, as well as emotional and psychological distress [Strutton et al., supra, 2004].

Hyperhidrosis is associated with the excessive functioning of the sympathetic nervous system, specifically the thoracic sympathetic ganglion chain that controls the sweat glands, with acetylcholine acting as the major neurotransmitter. Acetylcholine release from the sympathetic nerves stimulates postsynaptic muscarinic receptors present in the basolateral membrane of the eccrine gland cells, resulting in secretion of sweat to the skin surface. All of the 5 subtypes of muscarinic receptors have been identified in different anatomical locations of the human eccrine glands [Kurzen et al., J Invest Dermatol 2004; 123:937-949]. More specifically, myoepithelial cells express all of the 5 subtypes of muscarinic receptors, acinar cells express M1 (weakly), M3 and M4, while the sweat duct epithelium expresses M1 (weakly), M3, M4, and M5 subtypes.

Hyperhidrosis may be congenital or be an acquired trait. Hyperhidrosis may be categorized as being the result of an underlying health condition, or one with no apparent cause, such as in primary idiopathic hyperhidrosis. Secondary hyperhidrosis is generally referred to when a person sweats too much because of an underlying health condition, such as obesity, gout, menopause, a tumor, mercury poisoning, diabetes mellitus, psychiatric disorders or hyperthyroidism (overactive thyroid gland). It can also be caused by some medications. Generalized hyperhidrosis is more common among patients with secondary hyperhidrosis.

Treatment options for hyperhidrosis include topical aluminium chloride hexahydrate, tap water iontophoresis, intradermal injections of botulinum toxin type A (BTX-A), systemic anticholinergics, and endoscopic transthoracic sympathectomy. However, the usefulness of these treatments is limited. For example, topical aluminium chloride hexahydrate has limited efficacy and produces skin irritation at higher doses [Goh et al., Int J Dermatol. 1990; 29: 368-70 and Holze et al., Dermatologica 1987; 175: 126-135]; iontophoresis is a time consuming treatment [Karakoc, et al., Int J Dermatol. 2002; 41: 602-605 and Reinauer et al., Br J Dermatol 1993; 129: 166-169]; oral anticholinergics have substantial side effects (e.g., blurred vision, tachycardia, dry mouth, urinary retention, and constipation) [Bajaj et al., Br J Dermatol., 2007; 157: 118-121 and Gee et al., Thorac Surg Clin 2008; 18: 141-155]; BTX-A injections are associated with high cost, pain, muscular weakness, and slow onset of effect; and surgical procedures are associated with compensatory sweating and complications [Connolly et al., Am J Clin Dermatol 2003; 4: 681-697].

Topical anticholinergic agents can also potentially be used for the treatment of hyperhidrosis. For example, glycopyrrolate, a muscarinic anticholinergic agent, has been investigated and reported to be effective as a topical therapy in managing hyperhidrosis since 1978 [Hays et al. Laryngoscope 1978; 88: 1796-18241978; May et al., Head Neck 1989; 11: 85-89; Shaw et al., Diabetologia 1997; 40: 299-301 and Kim et al., Yonsei Med J 2003; 44: 579-582].

Other topical products for use in the treatment of hyperhidrosis include formaldehyde, potassium permanganate, glutaraldehyde and methenamine solution.

U.S. Pat. No. 6,433,003 describes methods for treating hyperhidrosis in humans by the topical administration of glycopyrrolate. U.S. Pat. Nos. 5,730,964 and 5,512,555 describe methods of treating sweat related conditions with 5-alpha-reductase inhibitors, such as finasteride, epristeride and cholestan-3-one, alone or in combination with other active agents to treat conditions such as apocrine gland sweating, hyperhidrosis and hydradenitis suppurativa.

U.S. Pat. No. 4,885,282 describes a method for the treatment of hyperhidrosis, ichthyosis or wrinkling, comprising applying to the affected area a compound selected from the group consisting of mono- and dicarboxylic acids having from 4 to 18 carbon atoms, a mercapto derivative thereof, a salt thereof, or an ester thereof.

US Patent Application No. 2005/0196414 describes a method of preventing or reducing symptoms associated with subjective or clinical hyperhidrosis, by topically applying a botulinum toxin to the skin or epithelium of a subject.

US Patent Application No. 2004/0192754 describes compounds that can ameliorate symptoms of idiopathic hyperhidrosis and associated conditions, such as 5-HT2C receptor antagonists (i.e., ketanserin, ritanserin, mianserin, mesulergine, cyproheptadine, fiuoxetine, mirtazapine, olanzapine and ziprasidone) and 5-HT2C receptor modulators (i.e., inverse agonists, partial agonists and allosteric modulators).

To date, no topical anticholinergic agents have achieved regulatory approval for the treatment of focal hyperhidrosis. Thus, the identification of potent, pan-active antagonists of muscarinic acetylcholine receptors that can be delivered directly to the sweat glands in both axilla and palm (which has a higher number of stratum corneum layers that might present higher barrier properties [Ya-Xian et al., Arch Dermatol Res 1999; 291: 555-559]) via topical dermal administration remains an unmet medical need. The present disclosure is believed to meet such needs by providing a pharmaceutical composition for the treatment of excessive sweating and/or hyperhidrosis in a patient.

### BRIEF DESCRIPTION OF THE DRAWINGS

FIG. 1 illustrates the cumulative amount of umeclidinium (ng) delivered into the receiving fluid. Time points collected hourly, from 0 to 24 hours. Time points represent the average of 12≦n≦17 replicates (4 skin donors). Error bars represent the standard error of the mean (SEM).

FIG. 2 illustrates the distribution of palm and axilla size in adult males based on palm and axilla measurements [Agarwal P. Sahu S. Indian Journal of Plastic Surgery 2010; 43: 49-53 and Cowan-Ellsberry C et al., Regul Toxicol Pharmacol 2008; 52: 46-52].

FIG. 3 illustrates the individual and combined distributions of the amount of formulation (g) applied using: 1) deodorants with clicks; 2) deodorants with turn knobs; and 3) invisible stick deodorants.

FIG. 4 illustrates the predicted pharmacokinetic profile of umeclidinium following topical administration of 165 mg of a 2.2% umeclidinium bromide (1.85% w/w umeclidinium cation) solution to the axilla (surface area of 40 cm²) for 8 hours.

FIG. 5 illustrates the population pharmacokinetic model structure describing the plasma concentrations following administration of umeclidinium either directly to the occluded axilla or as an intravenous bolus dose.

FIG. 6 illustrates simulated mean and 90% confidence interval of concentration time profiles following repeated once daily doses of umeclidinium applied to both axillas for 15 days.

FIG. 7 illustrates simulated mean and 90% confidence interval of concentration time profiles following repeated once weekly doses of umeclidinium applied to both axillas for 15 weeks.

FIG. 8 illustrates the cumulative amount of umeclidinium (ng) delivered into the receiving fluid. Time points collected hourly, from 0 to 24 hours. Time points represent the average of 20≦n≦25 replicates (3 skin donors). Error bars represent the standard error of the mean (SEM).

### SUMMARY OF THE DISCLOSURE

The present disclosure provides for the use of umeclidinium for treating any condition involving or promoting excessive sweating, typically involving the whole body, including hyperthyroidism or similar endocrine disorders, obesity and menopause. Thus, the treatment reduces or minimizes excessive sweating from what would naturally occur. Umeclidinium is suitable for treating, ameliorating or reducing perspiration, especially excessive sweating such as hyperhidrosis including palmar hyperhidrosis, axillary hyperhidrosis, plantar hyperhidrosis, hyperhidrosis of the trunk and/or the thighs, or the groin, and facial hyperhidrosis, and any combination of them. Suitably, the administration does not exceed 20% of the body surface area for one application, depending on the dose utilized.

Suitably, the treatment is for primary focal hyperhidrosis. In another embodiment, the treatment is for use on the axilla, the palms and/or the soles. In still another embodiment, the treatment is for axial use. In yet another embodiment, the treatment is for palmar usage, and in yet another embodiment, the treatment is for soles of the feet.

In one embodiment, the present disclosure provides for the novel use of umeclidinium for the topical treatment or prophylaxis of excessive sweating.

In another embodiment, the present disclosure provides a method for the treatment or prophylaxis of excessive sweating in a patient in need thereof with the method comprising administering a therapeutically effective amount of umeclidinium to the skin of the patient.

In yet another embodiment, the present disclosure provides a method for the treatment or prophylaxis of excessive sweating in a patient in need thereof with the method comprising administering a pharmaceutical composition comprising a therapeutically effective amount of umeclidinium and a pharmaceutically acceptable carrier to the skin of the patient. In one embodiment the a pharmaceutically acceptable carrier is a pharmaceutically acceptable solvent.

It is understood that the current clinical literature appears to conflict as to whether hyperhidrosis results in excessive sweating or whether excessive sweating is a species of hyperhidrosis, nonetheless the treatment of this disclosure addresses all excessive sweating and odors associated therewith or derived therefrom.

In a further embodiment, the present disclosure provides for the use of umeclidinium in the manufacture of a medicament for the topical treatment or prophylaxis of excessive sweating in a patient in need thereof.

Another embodiment of the present disclosure is 4-[Hydroxy(diphenyl)methyl]-1-{2-[(phenylmethyl)oxy]ethyl}-1-azoniabicyclo[2.2.2]octane and a pharmaceutically acceptable anion thereof (Umeclidinium) for use in the topical treatment or prophylaxis of excessive sweating. In one embodiment, the pharmaceutically acceptable anion is bromide.

Another embodiment of the present disclosure is a method for the treatment or prophylaxis of excessive sweating with the method comprising administering to a patient in need thereof, a first therapeutic agent that is umeclidinium and at least one other therapeutic agent. The administration can be simultaneous, or as successive administration of the first therapeutic agent and at least one other therapeutic agent (in any order). Suitably the second therapeutic agent is also administered topically. In one embodiment, the umeclidinium and the at least one other therapeutic agent are administered in the same pharmaceutical product.

In a further embodiment, the present disclosure provides a topical pharmaceutical composition comprising a therapeutically effective amount of umeclidinium, and at least one pharmaceutically acceptable solvent.

In an embodiment, the present disclosure provides a topical pharmaceutical composition comprising a therapeutically effective amount of umeclidinium, and at least one pharmaceutically acceptable solvent, and wherein the composition when applied topically to human skin, has a skin flux of at least 0.2 ng/cm²/hour measured in vitro using ex vivo human skin.

One embodiment of the present disclosure is a topical pharmaceutical composition comprising: a therapeutically effective amount of umeclidinium, at least one pharmaceutically acceptable solvent and a penetration enhancer.

One embodiment of the present disclosure is a topical pharmaceutical composition comprising: a therapeutically effective amount of umeclidinium; water, and a water misicible pharmaceutically acceptable solvent.

One embodiment of the present disclosure is a topical pharmaceutical composition comprising: a therapeutically effective amount of umeclidinium; water, a water misicible pharmaceutically acceptable solvent, and a penetration enhancer.

Another embodiment of the present disclosure is a topical pharmaceutical composition comprising: a therapeutically effective amount of umeclidinium; a pharmaceutically acceptable solvent, and optionally a chelating agent, a penetration enhancer, an antioxidant, a pH adjusting agent, and a gelling agent.

In one embodiment the composition is in the form of a solution, a gel, a cream, an ointment, a lotion, a spray, an aerosol spray or an aerosol foam. In another embodiment, the composition is a solution. In still another embodiment, the composition is a gel.

In one embodiment, the solvent comprises a mixture of water and a water miscible organic solvent. In another embodiment, the water is present in an amount from about 5% to about 55% by weight and the water miscible organic solvent is present in an amount from about 45% to about 90% by weight, based on the total weight of the composition.

Another embodiment of the present disclosure is a pharmaceutical composition for topical administration comprising a therapeutically effective amount of umeclidinium, and a pharmaceutically acceptable solvent, in which the composition produces an AUC(0-tau) at steady state of less than 2541 hr*pcg/mL.

Another embodiment of the present disclosure is a pharmaceutical composition for topical administration comprising a therapeutically effective amount of umeclidinium, and a pharmaceutically acceptable solvent, in which the composition produces a maximum plasma level of umeclidinium less than 1607 pcg/mL at steady state.

Another embodiment of the present disclosure is a pharmaceutical composition for topical administration comprising a therapeutically effective amount of umeclidinium, and a pharmaceutically acceptable solvent, in which the composition produces a maximum plasma level of umeclidinium less than 1607 pcg/mL, and an AUC(0-tau) at steady state of less than 2541 hr*pcg/mL.

In one embodiment, the umeclidinium composition is applied to the affected area(s) twice daily, once daily, once every second day, three times weekly, twice weekly or once weekly.

In one embodiment, the umeclidinium composition is applied in two phases to the affected area(s), by an initial dosing regimen which may be twice daily, or once daily, and then a maintenance phase which may be once every second day, three times weekly, twice weekly or once weekly.

### DETAILED DESCRIPTION OF THE DISCLOSURE

Umeclidinium is represented by the formula of compound (I) below:

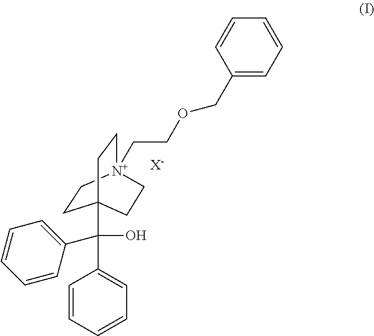

wherein X⁻ is a pharmaceutically acceptable anion.

One accepted chemical name of this structure is 4-[hydroxy(diphenyl)methyl]-1-{2-[(phenylmethyl)oxy]ethyl}-1-azoniabicyclo[2.2.2]octane, and a pharmaceutically acceptable anion.

Any suitable pharmaceutically acceptable anion of umeclidinium is acceptable for use in the invention. Suitably, the pharmaceutically acceptable anion is selected from chloride, bromide, iodide, hydroxide, sulfate, nitrate, phosphate, acetate, trifluoroacetate, fumarate, citrate, tartrate, oxalate, succinate, mandelate, methanesulfonate or p-toluenesulfonate.

For purposes herein, the term umeclidinium will generally appear without the terminology “and a pharmaceutically acceptable anion thereof” and represents inclusion of any suitable pharmaceutically acceptable anion. In some instances, such as for the purposes of calculating dosages, the umeclidinium cation (i.e. umeclidinium without the pharmaceutically acceptable anion) will be referred to herein.

However, if a particular pharmaceutically acceptable anion is contemplated, such as the bromide anion, the compound will be referred to as umeclidinium bromide. In another embodiment, if the pharmaceutically acceptable anion of umeclidinium is the iodide anion, the compound will be referred to as umeclidinium iodide. In yet another embodiment, if the pharmaceutically acceptable anion of umeclidinium is the chloride anion, the compound will be referred to as umeclidinium chloride, and so forth.

Umeclidinium is a potent long-acting pan-active muscarinic antagonist (LAMA). Notably, the compound has recently been approved by the FDA as a component of a fixed dose combination with vilanterol. The combination is known as Anoro™ Elipta®, which is an orally inhaled treatment for chronic obstructive pulmonary disease (COPD). The monotherapy has also been approved in Europe and the US as Incruse™ Elipta®.

Data available from the clinical development program of umeclidinium as inhalation therapy for COPD, have documented that umeclidinium is well tolerated following administration by several routes including oral, intravenous and oral inhalation. Data from these trials suggest that umeclidinium has “flip flop” pharmacokinetics (also referred to as absorption-rate limited pharmacokinetics), where the terminal phase after inhalation (and likely other routes excluding intravenous administration) represents the rate of absorption, not elimination.

The present disclosure provides for the novel use of umeclidinium for the topical treatment or prophylaxis of excessive sweating, which can be claimed as umeclidinium for use in the topical treatment or prophylaxis of excessive sweating or can be claimed as the use of umeclidinium in the manufacture of a medicament for the topical treatment or prophylaxis of excessive sweating.

The present disclosure also provides a method for the treatment or prophylaxis of excessive sweating in a patient in need thereof with the method comprising administering a therapeutically effective amount of umeclidinium to the skin of the patient.

The present disclosure provides for the treatment of any condition characterized by excessive sweating. In particular, the compounds and compositions of the present disclosure are suitable for treating, ameliorating or reducing hyperhidrosis. In an embodiment, the hyperhidrosis is selected from palmar hyperhidrosis, axillary hyperhidrosis, plantar hyperhidrosis, hyperhidrosis of the trunk and/or the thighs, and facial hyperhidrosis, and a combination thereof.

In one embodiment, the hyperhidrosis is axillary hyperhidrosis. In another embodiment, the hyperhidrosis is palmar hyperhidrosis.

In another embodiment, the present disclosure is directed to a method for the treatment or prophylaxis of excessive sweating in a patient in need thereof with the method comprising administering, either simultaneously or sequentially, to the patient: (i) a topical pharmaceutical composition comprising a therapeutically effective amount of umeclidinium, and (ii) at least one other pharmaceutical composition, vehicle or ingredient. Suitably, the at least one other pharmaceutical composition is also administered topically.

In yet another embodiment, the present disclosure is directed to a method for the treatment of, or minimization of or prophylaxis of undesirable odors associated with human sweat glands and skin in a patient in need thereof with the method comprising administering a therapeutically effective amount of umeclidinium to the skin of the patient.

Additional dose calculations were performed under the assumption that the average amount of API applied was between 0.78-4.02 mg/cm² (or alternatively ul/cm²) (based on the delivery characteristics of the device).

Suitably, a pharmaceutical composition comprising umeclidinium and a pharmaceutically acceptable solvent, is administered topically at a dose of from about 0.005 to about 10,000 mg/day, if necessary in divided doses, to at least one affected area. In another embodiment, the amount of umeclidinium is administered topically at a dose from about 0.005 to about 5000 mg daily. In another embodiment, the amount of umeclidinium is administered topically at a dose from about 0.005 to about 2500 mg daily. In another embodiment, the dose is from about 0.01 to about 2500 mg or about 0.01 to about 5000 mg daily.

In one embodiment, the compound in the pharmaceutical composition comprises umeclidinium bromide. In one embodiment, the composition can be presented for use in unit dose forms.

Where the treatment is to the axilla area, the total amount of umeclidinium administered topically in one dose is from about 0.02 to about 150 mg. Suitably, this administration is once daily. In another embodiment, the amount of umeclidinium administered topically is from about 0.01 to about 100 mg in one dosage administration. In yet another embodiment, the amount of umeclidinium administered is from about 0.03 to about 50 mg in one dosage administration

In another embodiment, where the treatment is to the palms, the total amount of umeclidinium administered topically is from about 0.02 to about 400 mg, in one dosage administration. In another embodiment, the total amount of umeclidinium administered topically to the palms is from about 0.02 to about 250 mg, in one dosage administration. In another embodiment, the amount of umeclidinium administered is from about 0.03 to about 150 mg in one dosage administration.

In yet another embodiment, where the treatment is to both the axilla and the palms, the total amount of umeclidinium administered topically is from about 0.02 to about 600 mg; or alternatively from about 0.03 to about 450 mg in one dosage administration. Suitably, the administration is once daily.

In another embodiment, the total amount of umeclidinium administered is from about 0.06 to about 300 mg. In another embodiment, the total amount of umeclidinium administered is from about 0.06 to about 200 mg per dosage administration.

In a further embodiment, the treatment is administered twice daily, to any affected area, the total amount of umeclidinium administered topically is from about 0.02 to about 1200 mg. In another embodiment, the total amount of umeclidinium administered is from about 0.03 to about 600 mg.

In another embodiment, the treatment is administered twice daily to both the axilla and the palms, and the total amount of umeclidinium administered topically daily is from about 0.02 to about 1200 mg. In another embodiment, the total amount of umeclidinium administered is from about 0.03 to about 600 mg daily.

In another embodiment, the treatment is administered twice daily to both the palms and the soles, the total amount of umeclidinium administered topically is from about 0.02 to about 1200 mg. In another embodiment, the amount of umeclidinium administered is from about 0.03 to about 600 mg daily.

In another embodiment, the treatment is administered twice daily to both the axilla and the soles, the total amount of umeclidinium administered topically is from about 0.02 to about 1200 mg. In another embodiment, the amount of umeclidinium administered is from about 0.03 to about 600 mg daily.

It is recognized that the additional administration of a dosage can be to the palms or soles, with or without additional treatment to the axilla or another body area.

In an embodiment, the dosing frequency to the affected area(s) can be twice daily, once daily, once every second day, three times weekly, twice weekly or once weekly, with the dose represented by any of the embodiments herein. In another embodiment, the treatment can be administered in two phases, an initial dosage frequency such as once daily, or twice daily, followed by a maintenance phase, such as every second day, three times weekly, twice weekly, or once weekly.

In an alternative method for calculation of the dosage to be applied to the skin, it is possible to use body surface area (BSA). The amount of umeclidinium administered topically can be based upon about 1% to about 20% of a BSA application.

Thus, in one embodiment, the amount of umeclidinium applied topically can be from about 0.01 to about 160 mg, based upon a 1% BSA. Calculations of the amounts will be based upon 1% BSA, such that the dose associated with a 20% BSA will be from about 0.2 to about 3200 mg. In one embodiment, the umeclidinium administered is umeclidinium bromide.

It is recognized that the dosage amount can vary depending upon the gender and size of the patient, and the amount of affected area, e.g. the body surface area to be treated.

In one embodiment, doses to be administered topically during a single administration ranges from about 0.01% to about 5% by weight of umeclidinium, based on the total weight of the composition. In another embodiment, doses to be administered topically range from about 0.01% to about 4% by weight of umeclidinium. In another embodiment, doses to be administered topically during a single administration ranges from about 0.01% to about 3% by weight of umeclidinium. In another embodiment, a dose to be administered topically up to about 1.85 (cation) % by weight of umeclidinium, or alternatively up to 2.2% as a bromide salt. This range can be applied to a maximum of 20% of the body surface area.

In another embodiment, the topical compositions of the present disclosure comprise umeclidinium in an amount from about 0.1% to about 5% by weight, based on the total weight of the composition. In another embodiment, the topical compositions of the present disclosure comprise umeclidinium in an amount from about 0.01% to about 4% by weight. In another embodiment, the topical compositions of the present disclosure comprise umeclidinium in an amount from about 0.01% to about 3% by weight. In another embodiment, the topical compositions of the present disclosure comprise umeclidinium in an amount up to about 1.85 (cation) % by weight. In another embodiment the topical compositions of the present disclosure comprise umeclidinium in an amount up to about 2.2% by weight of the bromide salt. Again, this range can be applied to a maximum of 20% of the body surface area.

In one embodiment, the amount of umeclidinium to be applied topically is to a maximum of 20% of the BSA of the patient. In one embodiment, the amount of umeclidinium to be applied topically is to a maximum of 10% of the BSA of the patient. In another embodiment, the amount of umeclidinium to be applied topically is to a maximum of 8% of the BSA of the patient. In yet another embodiment, the amount of umeclidinium to be applied topically is to a maximum of 4% of the BSA of the patient.

### Topical Pharmaceutical Compositions

The present disclosure also provides for a topical pharmaceutical composition comprising a therapeutically effective amount of umeclidinium, and a pharmaceutically acceptable solvent.

Examples of suitable topical pharmaceutical compositions according to the present disclosure include a solution, a gel, a cream, an ointment, a lotion, a spray, an aerosol spray, or an aerosol foam. In one embodiment, the topical pharmaceutical composition is a solution. In another embodiment, the topical pharmaceutical composition is a gel.

The compositions of the present disclosure can be applied to the skin by an applicator, such as a roll-on, stick, impregnated wipe or impregnated glove. The compositions of the present disclosure can also be dispensed from a pump pack or from an aerosol container (i.e., in the case of an aerosol spray or aerosol foam), for example.

Suitably, umeclidinium is present in an amount from about 0.1% to about 30% by weight, based on the total weight of the composition. In another embodiment, umeclidinium is present in an amount from about 0.1% to about 10% by weight, based on the total weight of the composition. In another embodiment, umeclidinium is present in an amount from about 0.5 to about 5% by weight, based on the total weight of the composition. In yet another embodiment, umeclidinium is present in an amount from about 1% to about 3% by weight, based on the total weight of the composition. In a further embodiment, umeclidinium is present in an amount of about 1% by weight, based on the total weight of the composition. In yet a further embodiment, umeclidinium is present in an amount of about 2.2% by weight. In an embodiment, umeclidinium is present in an amount of about 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0% by weight, based on the total weight of the composition.

### Solvent

The topical pharmaceutical compositions of the present disclosure comprises at least one pharmaceutically acceptable solvent. In one embodiment, the solvent comprises a mixture of two or more solvents.

In one embodiment, the solvent comprises a mixture of water and at least one water miscible organic solvent.

In another embodiment, the water is present in an amount from about 5% to about 95% by weight, based on the total weight of the composition. In another embodiment, the water is present in an amount from about 5% to about 60% by weight or about 5% to about 55% by weight, based on the total weight of the composition. In another embodiment, the water is present in an amount from about 5% to about 55% by weight, based on the total weight of the composition. In yet another embodiment, the water is present in an amount from about 5% to about 40% by weight, based on the total weight of the composition. In yet another embodiment, the water is present in an amount from about 5% to about 30% by weight, based on the total weight of the composition. In a further embodiment, the water is present in an amount from about 5% to about 25% by weight, based on the total weight of the composition.

In one embodiment, the water miscible organic solvent is a mixture of two or more water miscible organic solvents.

Exemplary water miscible organic solvents for use herein include, but are not limited to, one or more alcohols and one or more ethers, and mixtures thereof.

In one embodiment, the water miscible organic solvent is an alcohol. Exemplary alcohols include, but are not limited to, methyl alcohol, ethyl alcohol, isopropyl alcohol, n-propyl alcohol, isobutyl alcohol, n-butyl alcohol, t-butyl alcohol, benzyl alcohol, tetrahydrofurfuryl alcohol, butylene glycol, diethylene glycol, diethylene glycol monoethyl ether, dipropylene glycol, ethylene glycol, ethyl hexanediol, ethylene glycol, 1,2-hexanediol, hexylene glycol, pentylene glycol, propanediol and propylene glycol, and mixtures thereof. In another embodiment, the alcohol is diethylene glycol monoethyl ether. In another embodiment, the alcohol is propylene glycol. In another embodiment, the alcohol is benzyl alcohol. In another embodiment, the water miscible organic solvents are propylene glycol and diethylene glycol monoethyl ether. In another embodiment the water miscible organic solvents are propylene glycol, diethylene glycol monoethyl ether and isopropyl alcohol. In another embodiment the water miscible organic solvents are propylene glycol, diethylene glycol monoethyl ether and ethanol. In another embodiment the water miscible organic solvents are propylene glycol, diethylene glycol monoethyl ether and isopropyl alcohol.

In one embodiment, the water miscible organic solvent is a mixture of two alcohols, diethylene glycol monoethyl ether and propylene glycol. In another embodiment, the water miscible organic solvent is a mixture of three alcohols. In still another embodiment, the mixture of three alcohols is diethylene glycol monoethyl ether, propylene glycol and benzyl alcohol.

In an embodiment, the ratio of diethylene glycol monoethyl ether to propylene glycol is from about 1:0.5 to about 1:4. In another embodiment, the ratio of diethylene glycol monoethyl ether to propylene glycol is from about 1:1 to about 1:3. In yet another embodiment, the ratio of diethylene glycol monoethyl ether to propylene glycol is about 1:1. In a further embodiment, the ratio of diethylene glycol monoethyl ether to propylene glycol is about 1:2, 1:2.1, 1:2.2, 1:2.3, 1:2.4 or 1:2.5. In yet a further embodiment, the ratio of diethylene glycol monoethyl ether to propylene glycol is about 1:2.3.

In an embodiment, the solvent comprises a mixture of water, and two alcohols. In one embodiment, the solvent mixture comprises water, diethylene glycol monoethyl ether and propylene glycol. Suitably, the ratio of water, diethylene glycol monoethyl ether and propylene glycol is from about 1:1:1 to about 1:2:4.5.

In a further embodiment, the water miscible organic solvent is a mixture of ethanol and propylene glycol. In yet a further embodiment, the water miscible organic solvent is a mixture of ethanol, propylene glycol and diethylene glycol monoethyl ether.

In another embodiment, the water miscible organic solvent is an ether. Examples of suitable polyethylene glycols include polyethylene glycol 200, polyethylene glycol 300, polyethylene glycol 400, polyethylene glycol 540, polyethylene glycol 600, polyethylene glycol 900, polyethylene glycol 1000, polyethylene glycol 1450, polyethylene glycol 1500, polyethylene glycol 1540, polyethylene glycol 1600, polyethylene glycol 3350, polyethylene glycol 4000, polyethylene glycol 6000 and polyethylene glycol 8000, and mixtures thereof.

Exemplary ethers include, but are not limited to, benzyl glycol, dimethyl isosorbide and a polyethylene glycol, and mixtures thereof. In another embodiment the ether is dimethyl isosorbide.

In another embodiment the water miscible organic solvents are dimethyl isosorbide, propylene glycol, diethylene glycol monoethyl ether and isopropyl alcohol and/or ethanol. Suitably, the water miscible organic solvent is present in the composition in an amount by weight from about 5% to about 95%. In one embodiment the water miscible organic solvent is present in the composition in an amount by weight from about 40% to about 95%. In another embodiment, the water miscible organic solvent is present in the composition in an amount from about 45% to about 90% by weight (based on the total weight of the composition). In another embodiment, the water miscible organic solvent is present in the composition in an amount from about 70% to about 90% by weight, based on the total weight of the composition.

The mixture of water and at least one water miscible organic solvent can further comprise a water immiscible organic solvent. That is, in one embodiment, the solvent mixture comprises a mixture of water, a water miscible organic solvent and a water immiscible pharmaceutically acceptable organic solvent.

Exemplary pharmaceutically acceptable water immiscible organic solvents include, but are not limited to, esters, such as coco-caprylate/caprate, diethyl sebacate, diisopropyl adipate, diisopropyl dilinoleate, ethyl oleate, isopropyl isostearate, isopropyl myristate, isopropyl palmitate and oleyl oleate, and mixtures thereof.

When a water immiscible organic solvent is used in combination with water and a water miscible organic solvent, the water immiscible organic solvent is present in an amount from about 1% to about 10% by weight, based on the total weight of the composition.

In another embodiment, the pharmaceutically acceptable solvent is a water miscible organic solvent, as described herein. In this embodiment, the water miscible organic solvent is present in an amount from about 70% to about 99.9% by weight, based on the total weight of the composition.

In yet another embodiment, the pharmaceutically acceptable solvent comprises a mixture of a water miscible organic solvent and a water immiscible organic solvent, as described herein. Together, the water miscible organic solvent and the water immiscible organic solvent are present in the composition in an amount from about 70% to about 99.9% by weight, based on the total weight of the composition.

In a further embodiment, the solvent is a water immiscible organic solvent. In this embodiment, the water immiscible organic solvent is present in an amount from about 70% to about 99.9% by weight, based on the total weight of the composition.

In another embodiment, the compositions of the present disclosure are free or substantially free of ethyl alcohol.

### Penetration Enhancer

The present topical pharmaceutical compositions can further comprise a penetration enhancer. The penetration enhancer can be in addition to the water miscible organic solvent and/or water immiscible organic solvents described herein that can act as both a solvent and a penetration enhancer. In an embodiment, the penetration enhancer is a mixture of two or more penetration enhancers. It is believed that the penetration enhancer also serves to enhance the solubility of the umeclidinium with the solvent.

Exemplary penetration enhancers include, but are not limited to, fatty acids, fatty acid esters, fatty alcohols, pyrrolidones, sulfoxides, alcohols, diols and polyols, and mixtures thereof.

Suitably, the penetration enhancer is present in the composition in an amount from about 1% to about 20% by weight, based on the total weight of the composition.

In an embodiment, the present disclosure provides a topical pharmaceutical composition comprising a therapeutically effective amount of umeclidinium, a solvent, and a penetration enhancer.

### Additional Pharmaceutically Acceptable Excipients

The topical pharmaceutical compositions of the present disclosure comprise one or more additional pharmaceutically acceptable excipients.

Suitably, the additional pharmaceutically acceptable excipient can be a gelling agent, a pH adjusting agent, a chelating agent, an antioxidant, a preservative, an antiperspirant, a deodorant, a fragrance, a humectant, a skin conditioning agent, a film forming agent, a plasticizer, a surfactant and a propellant, and a combination or mixture thereof. In one embodiment, the pharmaceutically acceptable excipient is a gelling agent, a penetration enhancer, a pH adjusting agent, a chelating agent, an antioxidant and a preservative, and a combination or mixture thereof.

### Gelling Agent

Again, the topical pharmaceutical compositions of the present disclosure can further comprise a gelling agent. In an embodiment, the gelling agent is a mixture of two or more gelling agents.

Exemplary gelling agents include, but are not limited to, cellulosic derivatives such as hydroxyethylcellulose (HEC), carboxymethylcellulose, hydroxypropylcellulose (HPC), and hydroxypropyl methylcellulose (HPMC); carbomers, acrylate copolymers, silica, polyvinylpyrrolidone (PVP), poloxamer, salts thereof, and a combination or mixture thereof.

In an alternative embodiment, the gelling agent is a small molecule gelling agent. Exemplary small molecule gelling agents include, but are not limited to, dibutyl ethylhexanoyl glutamide and dibutyl lauroyl glutamide.

In another embodiment, the gelling agent is a small molecule gelling agent, a cellulosic derivative such as hydroxyethylcellulose (HEC), carboxymethylcellulose, hydroxypropylcellulose (HPC), and hydroxypropyl methylcellulose (HPMC); carbomers, acrylate copolymers, silica, polyvinylpyrrolidone (PVP), poloxamer, salts thereof, and a combination or mixture thereof.

In one embodiment, the gelling agent is a carbomer or a salt thereof. Carbomer is a term used for a series of polymers made primarily from acrylic acid, e.g. sodium acrylate or a polyacrylates. Suitable carbomers are produced by Lubrizol under Carbopol® line such as Carbopol® 940 or 941. Carbomers are also measured similarly to the cellulosic derivative using a Brookfield viscosity of 25° C., and the like. The Carbopol polymers have high molecular weights ranging from about 250,000 to about 4,000,000. The viscosity of the final gel is dependent upon the polymer molecular weight, as well as the concentration of the polymer in the formulation. Carbomers as gelling agents are generally present in an amount from 0.1 to 5% (w/w). The higher molecular weight (mw) carbomers can be present in amounts from about 0.1 to about 1% w/w.

In another embodiment, the gelling agent is a cellulosic derivative, suitably hydroxypropylcellulose (HPC). Hydroxypropylcellulose is commercially available from many sources, such as from the Aqualon division Hercules Incorporated, Wilmington, Del., USA (or Dow Chemical Company) which markets HPC under the trade name Klucel®. Klucel is available as a pharmaceutical grade with at least six different viscosity types—HF, MF, GF, JF, LF and EF (having a molecular weight (MW) from about 1,150,000 to 80,000). Alternatively, the HXF, MXF, GXF, JXF, LXF and EXF grades, (course to fine particle sizes) may be used. These 6 grades have viscosity ranging from 75- to 6500 cps. In one embodiment, Klucel MF or MXF (MW 850,000) is used as a gelling agent in the compositions of the present invention and has a viscosity of 4,000 to 6,500 centipoise (cps) (measured at 25° C.) at a concentration of 2% in water.

It is recognized in the art that the determination of the viscosity of cellulosic derivatives is based upon standard techniques and grading in the art e.g. for HPC, viscosity may be determined at 25° C. using a Brookfield LVF viscometer with spindle and speed combination depending upon viscosity level as may be suggested by the manufacturer Hercules.

It is recognized that combinations of various molecular weight HPC's can be used to obtain a wide range of resulting viscosities. All such variations are encompassed within the scope of this invention.

In one embodiment, the gelling agent is polyvinylpyrrolidone (PVP).

In another embodiment, the gelling agent is a combination of polyvinylpyrrolidone (PVP) and at least one cellulosic derivative. Suitably, the cellulosic derivative is hydroxypropylcellulose.

In an alternative embodiment, the gelling agent is a small molecule gelling agent. Exemplary small molecule gelling agents include, but are not limited to, dibutyl ethylhexanoyl glutamide and dibutyl lauroyl glutamide.

Suitably, the gelling agent is present in the composition in an amount from about 0.1% to about 5% by weight, based on the total weight of the composition. In another embodiment, the gelling agent is present in the composition in an amount from about 0.1% to about 2% by weight, based on the total weight of the composition.

In one embodiment, the topical pharmaceutical composition is a gel and the viscosity of the resulting gel is from about 2,500 to about 100,000 centipoise at 25° C. In another embodiment, the viscosity of the gel is from about 5,000 to about 65,000 centipoise at 25° C. In another embodiment, the viscosity of the gel is from about 18,000 to about 55,000 centipoise at 25° C. In another embodiment, the viscosity of the gel is from about 25,000 to about 62,000 centipoise at 25° C.

### pH Adjusting Agent

The present topical pharmaceutical compositions can further comprise a pH adjusting agent.

In an embodiment, the pH adjusting agent is an acid, an acid salt, or a mixture thereof. Suitably, the acid is a lactic acid, acetic acid, maleic acid, succinic acid, citric acid, benzoic acid, boric acid, sorbic acid, tartaric acid, edetic acid, phosphoric acid, nitric acid, ascorbic acid, dehydroacetic acid, malic acid, propionic acid, sulphuric acid and hydrochloric acid, and mixtures thereof.

In another embodiment, the pH adjusting agent is a base. Suitably, the base is an aminomethylpropanol, diisopropanolamine, ethylenediamine, trolamine and tromethamine, and mixtures thereof.

In yet another embodiment, the pH adjusting agent is a buffer. Suitably, the buffer is citrate/citric acid, acetate/acetic acid, phosphate/phosphoric acid, propionate/propionic acid, lactate/lactic acid, ammonium/ammonia and edetate/edetic acid. In a particular embodiment, the pH adjusting agent is a buffer that is citrate/citric acid.

Suitably, the pH adjusting agent is present in the composition in an amount from about 0.01% to about 10% by weight, based on the total weight of the composition. In an embodiment of the present disclosure where the composition comprises water, the apparent pH of the composition is adjusted with a pH adjusting agent, suitably to an apparent pH of from about 3 to about 9. In one embodiment, the apparent pH of the composition is adjusted with a pH adjusting agent to an apparent pH of from about 4 to about 8.

### Chelating Agent

The present topical pharmaceutical compositions can further comprise a chelating agent. In an embodiment, the chelating agent is a mixture of two or more chelating agents. The compositions of the present disclosure can comprise a mixture of a chelating agent and an antioxidant, where both excipients act to prevent or minimize oxidative degradation reactions in the composition.

Exemplary chelating agents include, but are not limited to, citric acid, glucuronic acid, fumaric acid, malic acid, sodium hexametaphosphate, zinc hexametaphosphate, ethylene diamine tetraacetic acid (EDTA), phosphonates, salts thereof, and mixtures thereof. Ethylene diamine tetraacetic acid is also known as edetic acid.

In one embodiment, the chelating agent is EDTA or a salt thereof, such as potassium, sodium or calcium salts of EDTA. In another embodiment, the chelating agent is citric acid.

Suitably, the chelating agent is present in the composition in an amount from about 0.01% to about 1% by weight, based on the total weight of the composition. The chelating agent may be used to prevent or minimize oxidative degradation reactions within the composition.

### Antioxidant

The present topical pharmaceutical compositions can further comprise an antioxidant. In an embodiment, the antioxidant is a mixture of two or more antioxidants.

Exemplary antioxidants include, but are not limited to, butylated hydroxytoluene (BHT), butylated hydroxyanisole (BHA), tocopherol, propyl gallate, vitamin E TPGS and tert-Butylhydroquinone (TBHQ), and mixtures thereof.

In one embodiment, the antioxidant is butylated hydroxytoluene. In another embodiment, the antioxidant is propyl gallate. In yet another embodiment, the antioxidant is mixture of butylated hydroxytoluene and propyl gallate.

Suitably, the antioxidant is present in the composition in an amount from about 0.001% to about 1% by weight, based on the total weight of the composition.

### Preservative

The present topical pharmaceutical compositions can further comprise a preservative. In an embodiment, the preservative is a mixture of two or more preservatives.

Exemplary preservatives include, but are not limited to, benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, salts thereof, and mixtures thereof.

In one embodiment, the preservative is benzyl alcohol. In another embodiment, the preservative is phenoxyethanol.

Suitably, the preservative is present in the composition in an amount from about 0.01% to about 2% by weight, based on the total weight of the composition.

One embodiment of the present disclosure is a topical pharmaceutical composition comprising:
a therapeutically effective amount of umeclidinium,
at least one pharmaceutically acceptable solvent,
a penetration enhancer
and optionally, one or more of: a chelating agent, an antioxidant, a pH adjusting agent, and a gelling agent.

One embodiment of the present disclosure is a topical pharmaceutical composition comprising:
a therapeutically effective amount of umeclidinium,
water,
at least one water miscible pharmaceutically acceptable solvent,
a penetration enhancer, and
optionally one or more of a chelating agent, an antioxidant, a pH adjusting agent, and a gelling agent.

In an embodiment, the present disclosure provides a topical pharmaceutical composition comprising:
a) umeclidinium present in an amount from about 0.5% to about 5% by weight;
b) water in an amount from about 5% to about 55% by weight; and
c) at least one water miscible organic solvent in an amount from about 45% to about 90% by weight,
   P wherein all % are based on the total weight of the composition.

In another embodiment, the present disclosure provides a topical pharmaceutical composition comprising:
a) umeclidinium present in an amount from about 0.5% to about 5% by weight;
b) water in an amount from about 5% to about 55% by weight; and
c) at least one water miscible organic solvent in an amount from about 45% to about 90% by weight,
   wherein all % are based on the total weight of the composition and the composition is a solution.

In yet another embodiment, the present disclosure provides a topical pharmaceutical composition comprising:
a) umeclidinium present in an amount from about 0.5% to about 5% by weight;
b) water in an amount from about 5% to about 55% by weight;
c) at least one water miscible organic solvent in an amount from about 45% to about 90% by weight; and
d) a gelling agent in an amount from about 0.1% to about 5% by weight, and
   wherein all % are based on the total weight of the composition and the composition is a gel.

In still another embodiment, the present disclosure provides a topical pharmaceutical composition comprising:
a) umeclidinium present in an amount from about 0.5% to about 5% by weight;
b) water in an amount from about 5% to about 25% by weight; and
c) at least one water miscible organic solvent in an amount from about 70% to about 90% by weight,
   wherein all % are based on the total weight of the composition.

In another embodiment, the present disclosure provides a topical pharmaceutical composition comprising:
a) umeclidinium present in an amount from about 0.5% to about 5% by weight;
b) water in an amount from about 5% to about 25% by weight; and
c) at least one water miscible organic solvent in an amount from about 70% to about 90% by weight,
   wherein all % are based on the total weight of the composition and the composition is a solution.

In yet another embodiment, the present disclosure provides a topical pharmaceutical composition comprising:
a) umeclidinium present in an amount from about 0.5% to about 5% by weight;
b) water in an amount from about 5% to about 25% by weight;
c) at least one water miscible organic solvent in an amount from about 70% to about 90% by weight; and
d) a gelling agent in an amount from about 0.1% to about 5% by weight, and
   wherein all % are based on the total weight of the composition and the composition is a gel.

In a further embodiment, the present disclosure provides a topical pharmaceutical composition comprising:
a) umeclidinium present in an amount from about 0.5% to about 5% by weight;
b) water in an amount from about 5% to about 25% by weight; and
c) at least one water miscible organic solvent in an amount from about 70% to about 90% by weight, which is a mixture comprising diethylene glycol monoethyl ether and propylene glycol, and
   wherein all % are based on the total weight of the composition.

In yet a further embodiment, the present disclosure provides a topical pharmaceutical composition comprising:
a) umeclidinium present in an amount from about 0.5% to about 5% by weight;
b) water in an amount from about 5% to about 25% by weight; and
c) at least one water miscible organic solvent in an amount from about 70% to about 90% by weight, which is a mixture comprising diethylene glycol monoethyl ether, propylene glycol and benzyl alcohol, and
   wherein all % are based on the total weight of the composition.

In an embodiment, the invention provides a topical pharmaceutical composition comprising a therapeutically effective amount of umeclidinium, and at least one pharmaceutically acceptable solvent, wherein the composition when applied topically to human skin, has a skin flux of at least 0.2 ng/cm²/hour measured in vitro using ex vivo human skin.

In an embodiment, the present disclosure provides a topical pharmaceutical composition comprising a therapeutically effective amount of umeclidinium, water, and at least one water miscible organic solvent, and wherein the composition has a skin flux of at least 0.2 ng/cm²/hour measured in vitro using ex vivo human skin.

In another embodiment, the present disclosure provides a topical pharmaceutical composition comprising a therapeutically effective amount of umeclidinium, a pharmaceutically acceptable solvent comprising a mixture of a water miscible organic solvent and a water immiscible organic solvent, and wherein the composition has a skin flux of at least 0.2 ng/cm²/hour measured in vitro using ex vivo human skin.

In a further embodiment, the composition has an average skin flux of at least 0.2 ng/cm²/hour measured in vitro using ex vivo human abdominal skin. In yet a further embodiment, the composition has a skin flux of at least 0.5 ng/cm²/hour measured in vitro using ex vivo human abdominal skin. In yet a further embodiment, the composition has a skin flux of at least 1 ng/cm²/hour measured in vitro using ex vivo human abdominal skin. In an embodiment, the composition has a skin flux of at least 2 ng/cm²/hour measured in vitro using ex vivo human abdominal skin. In another embodiment, the composition has a skin flux of at least 3 ng/cm²/hour measured in vitro using ex vivo human abdominal skin. In yet another embodiment, the composition has a skin flux of at least 4 ng/cm²/hour measured in vitro using ex vivo human abdominal skin. In a further embodiment, the composition has a skin flux of at least 5 ng/cm²/hour measured in vitro using ex vivo human abdominal skin.

In yet a further embodiment, the composition has a skin flux from about 0.2 to about 8 ng/cm²/hour measured in vitro using ex vivo human abdominal skin. In an embodiment, the composition has a skin flux from about 2.5 to about 7.5 ng/cm²/hour measured in vitro using ex vivo human skin. In another embodiment, the composition has a skin flux from about 5 to about 7 ng/cm²/hour measured in vitro using ex vivo human skin.

In one embodiment, the present disclosure provides a topical pharmaceutical composition comprising a therapeutically effective amount of umeclidinium, in which the composition when applied to ex vivo human skin in vitro results in a skin flux rate of umeclidinium relative to a topical solution of glycopyrrolate at equipotent molar doses, such that the in vitro skin flux for glycopyrrolate is 1.0 to 65-fold higher than that of the umeclidinium composition.

In another embodiment, the present disclosure provides a topical pharmaceutical composition comprising a therapeutically effective amount of umeclidinium having an in vitro transdermal flux (nmol/cm²/hr) equal to or less than that of glycopyrrolate, with a lower amount of unbound systemic exposure than glycopyrrolate.

In yet another embodiment, the present disclosure provides a topical pharmaceutical composition comprising a therapeutically effective amount of umeclidinium having a 6 hour cumulative amount (nmol) after an in vitro skin penetration study equal to or less than glycopyrrolate, but having a lower amount of unbound systemic exposure than glycopyrrolate.

The composition of Formulation 1 showed absorption in humans characterized by two sequential absorption processes: a zero order, followed by a first order with lag time, in which the zero order rate estimate is about 1.5 ng/cm²/hour (range: 0.16-12.9 ng/cm²/hour) representing the in vivo skin flux, and the first order rate constant estimate is 0.0656 (28.1%) hour⁻¹, likely representing absorption after washing at the site of application. In one aspect of the present disclosure is a formulation characterized by these same criteria. In an embodiment, the composition has a human in vivo skin flux from about 0.1 to about 40 ng/cm²/hour. In an embodiment, the composition has a human in vivo skin flux from about 0.01 to about 4 ng/cm²/hour. In an embodiment, the composition has a human in vivo skin flux from about 0.01 to about 40 ng/cm²/hour.

In another embodiment, a composition of the present disclosure produces a maximum plasma level of umeclidinium less than 1607 pcg/mL at steady state in a human upon topical administration.

In another embodiment, a composition of the present disclosure produces an AUC(0-tau) at steady state of less than 2541 hr*pcg/mL, in a human upon topical administration.

In another embodiment, a composition of the present disclosure produces a maximum plasma level of umeclidinium of less than 1607 pcg/mL, and an AUC(0-tau) at steady state of less than 2541 hr*pcg/mL, in a human upon topical administration.

In another embodiment, the absolute plasma bioavailability following application to one axilla is about 0.18% of the applied dose in humans, with a Cmax less than 1607 pcg/mL, an AUC less than 2541 hr*pcg/mL. In another embodiment, the absolute plasma bioavailability following application to the axilla is up to about 20% of the applied dose in humans, with a Cmax less than 1607 pcg/ml, and an AUC less than 2541 hr*pcg/mL. In one embodiment, the absolute plasma bioavailability following application to one axilla is less than 15% of the applied dose in humans, with a Cmax less than 1607 pcg/ml, and an AUC less than 2541 hr*pcg/mL at steady state. In an embodiment, the absolute plasma bioavailability following application to one axilla is less than 10% of the applied dose in humans, with a Cmax less than 1607 pcg/ml, and an AUC less than 2541 hr*pcg/mL at steady state. In one embodiment, the absolute plasma bioavailability following application to one axilla is less than 5% of the applied dose in humans with a Cmax less than 1607 pcg/ml, and an AUC less than 2541 hr*pcg/mL at steady state. In another embodiment, the absolute plasma bioavailability following application to one axilla is less than 1% of the applied dose in humans, with a Cmax less than 1607 pcg/ml, and an AUC less than 2541 hr*pcg/mL.

In one embodiment, the absolute plasma bioavailability following application to one axilla is less than 20% of the applied dose in humans.

In another embodiment, the absolute bioavailability following application to the palm is from up to 50%.

In yet another embodiment, the topical solution for transdermal delivery of umeclidinium bromide results in about 20 to 100% receptor occupancy (of the muscarinic receptors) following topical administration.

The present disclosure also provides a topical pharmaceutical composition comprising a therapeutically effective amount of umeclidinium, a pharmaceutically acceptable solvent, and a second pharmaceutically active agent.

The second pharmaceutically active agent is an agent suitable for use in treating excessive sweating such as: boric acid; tannic acid; resorcinol; potassium permanganate; formaldehyde; glutaraldehyde and methenamine; a Lewis acid; a salt or a complex of a metal or metal ion such as aluminum and zirconium; a 5-alpha-reductase inhibitor; finasteride; fiutamide; spironolactone; saw palmetto extract; epristeride; cholestan-3-one; mono- and/or dicarboxylic acids having from 4 to 18 carbon atoms, a mercapto derivative thereof, a salt thereof, or an ester thereof; botulinum toxin; a 5-HT2C receptor antagonist, such as ketanserin, ritanserin, mianserin, mesulergine, cyproheptadine, fiuoxetine, mirtazapine, olanzapine, ziprasidone; and a 5-HT2C receptor modulator; or an antiperspirant, such as aluminum chloride hexahydrate.

The compositions of the present disclosure can also further comprise as a second therapeutically effective agent a wound healing agent, a skin protective agent, a disinfectant or an anesthetic, alone or in combination with a second agent for treating hyperhidrosis.

### Synthetic Chemistry

The synthesis of umeclidinium bromide is described in Example 84 of WO 2005/104745, of which Example 84 is incorporated herein by reference.

Suitable pharmaceutically acceptable anions, such as the chloride, bromide or iodide anion can also be made according to the schemes as shown in WO2005/104745 with 2-bromo ethyl phenylmethyl ether or 2-iodo ethyl phenylmethyl ether, and the like.

### DEFINITIONS

The phrase “therapeutically effective amount” is used herein to refer to an amount of the umeclidinium sufficient to have a therapeutic effect upon administration. Effective amounts will vary with the particular condition being treated, the severity of the condition, the duration of the treatment, and the specific components of the composition. An effective amount of umeclidinium for treatment of excessive sweating can be determined by clinical techniques.

The terms “administering” and “administration” are used herein to mean any method which in sound medical practice delivers the pharmaceutical composition to a patient in such a manner as to provide the desired therapeutic effect.

As discussed above, the terms “body surface area” or “BSA” as used herein means the total surface area of the human body. A number of different formulas have been developed over the years to calculate the body surface area and they give slightly different results. A commonly used formula is that of Mosteller, published in 1987 in The New England Journal of Medicine. According to Mosteller's “simplified calculation of body-surface area in metric terms” the body surface area=the square root of product of the weight in kg times the height in cm divided by 3600. It is recognized that other formulas producing similar calculations are all included within the scope of this invention and are all within the skill of the practitioner for dosage administration.

As used herein, “topical” administration of the pharmaceutical composition refers to application of the composition to, and diffusion through, the stratum corneum.

The terms “treatment” or “treating” of excessive sweating encompasses alleviation of at least one symptom thereof, a reduction in the severity thereof, or the delay, prevention or inhibition of the progression thereof. Treatment need not mean that the condition or disorder is totally cured. A useful pharmaceutical composition herein need only to reduce the severity of the condition or disorder, reduce the severity of symptoms associated therewith, provide improvement to a patient's quality of life, or delay, prevent or inhibit the onset of the condition or disorder. A treatment need not be effective in every member of a population to have clinical utility, as is recognized in the medical and pharmaceutical arts.

The term “pharmaceutically acceptable” means approvable by a regulatory agency or listed in a Pharmacopeia or other generally recognized guide for use in animals, and more particularly in humans.

As used herein, the term “skin penetration” refers to the diffusion of the umeclidinium through the stratum corneum and into the epidermis and/or dermis of the skin or the systemic circulation.

As used herein, “patients” includes human patients.

As used herein, “substantially free” of a specified component refers to a composition with less than about 1% by weight of the specified component. “Free” of a specified component refers to a composition where the specified component is absent.

Any concentration range, percentage range or ratio range recited herein is to be understood to include concentrations, percentages or ratios of any integer within that range and fractions thereof, such as one tenth and one hundredth of an integer, unless otherwise indicated. This interpretation should apply regardless of the breadth of the range or the characteristic being described.

Unless otherwise indicated, all percentages are based on the percentage by weight, e.g. w/w of the final composition prepared, and all totals equal 100% by weight.

It should be understood that the terms “a” and “an” as used herein refer to “one or more” or “at least one” of the recited components. It will be clear to one of ordinary skill in the art that the use of the singular includes the plural unless specifically stated otherwise.

Throughout the specification, descriptions of various embodiments use “comprising” language, however in some specific instances, an embodiment can alternatively be described using the language “consisting essentially of” or “consisting of”.

All numbers expressing quantities, percentages or proportions, and other numerical values used in the specification, are to be understood as being modified in all instances by the term “about”.

As the biological profile (i.e. the pharmacokinetics (PK)/pharmacodynamics (PD)) of umeclidinium depends on the absence of degradation products and delivery of the active ingredient to the appropriate layers of the skin in efficacious amounts, compositions such as described herein offer patients a novel therapeutic treatment option for excessive sweating.

Other terms used herein are intended to be defined by their well-known meanings in the art.

### EXAMPLES

### Example 1

### Pharmaceutical Compositions

The following compositions were prepared as shown in the table below.

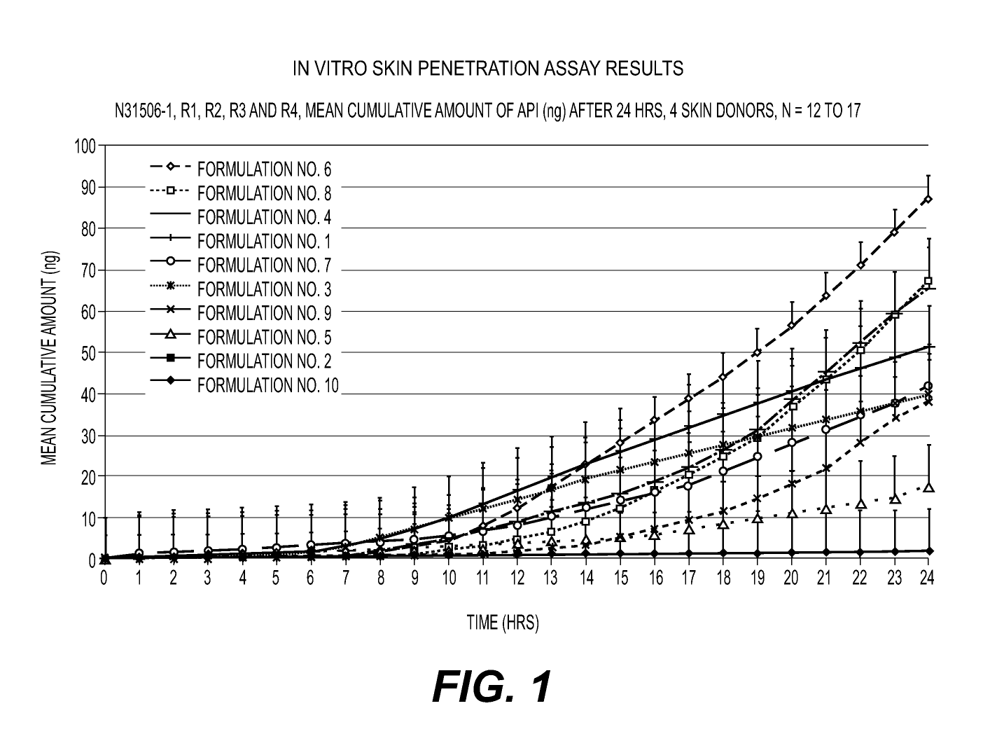

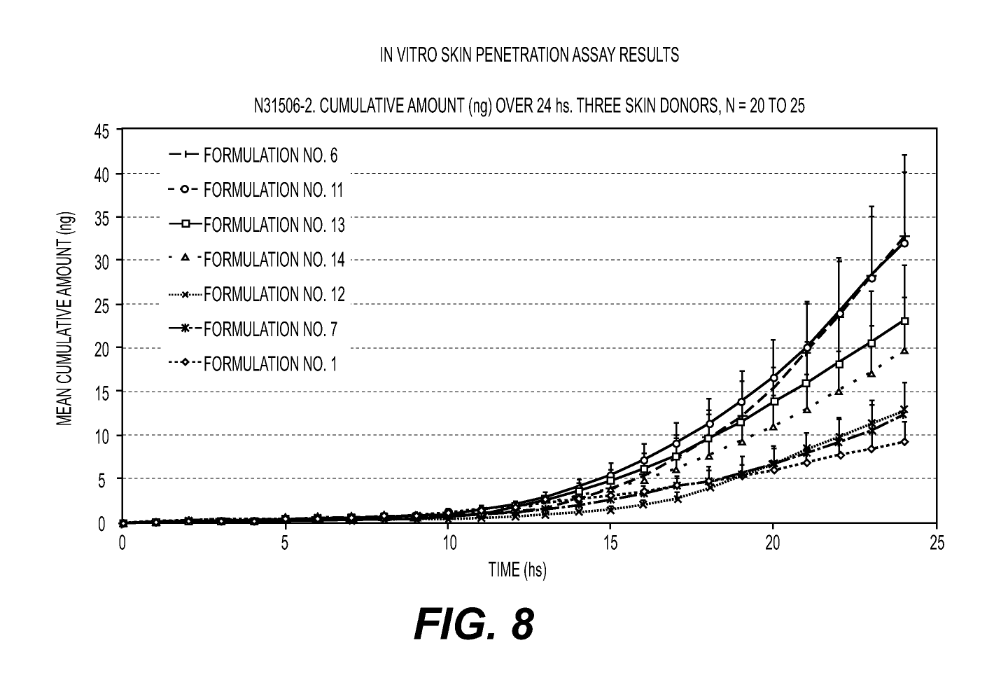

TABLE 1

| | Formula No. | | | | | | | | | |
|---|---|---|---|---|---|---|---|---|---|---|
| | 1 | 2 | 3 | 4 | 5 | 6 | 7 | 8 | 9 | 10 |
| Component % w/w | % | % | % | % | % | % | % | % | % | % |
| Diisopropyl adipate | 5 | 5 | 5 | — | — | — | — | — | — | — |
| Dimethyl isosorbide | 10 | 10 | — | — | — | — | — | — | — | — |
| Diethylene glycol | 25 | 25 | 25 | 25 | 25 | 25 | 25 | 25 | 25 | — |
| monoethyl ether | | | | | | | | | | |
| Propylene glycol | 26 | 26 | 26 | 42 | 42 | 56.4 | 56.4 | 57.6 | 57.6 | 25 |
| Benzyl alcohol | 1 | 1 | — | — | — | 1 | 1 | 1 | 1 | — |
| Isopropyl alcohol | 15.4 | 14.4 | 20.9 | 15.4 | 14.4 | — | — | — | — | — |
| Ethyl alcohol | — | — | — | — | — | — | — | — | — | 52.8 |
| Purified water | 15.4 | 14.4 | 20.9 | 15.4 | 14.4 | 15.4 | 13.4 | 15.4 | 13.4 | 19.2 |
| Umeclidinium | 2.2 | 2.2 | 2.2 | 2.2 | 2.2 | 2.2 | 2.2 | 1.0 | 1.0 | 1.0 |
| bromide | | | | | | | | | | |
| Hydroxypropyl- | — | 2 | — | — | 2 | — | 2 | — | 2 | 1.75 |
| cellulose | | | | | | | | | | |
| (Klucel-MF) | | | | | | | | | | |
| Polyvinyl | — | — | — | — | — | — | — | — | — | 0.25 |
| pyrolidone | | | | | | | | | | |
| TOTAL | 100 | 100 | 100 | 100 | 100 | 100 | 100 | 100 | 100 | 100 |
| Flux (ng/cm²/hour) | 3 | neg. | 2.2 | 5.7 | 1.3 | 5.9 | 2.9 | 6.5 | 4.3 | neg. |
| (data from FIG. 1) | | | | | | | | | | |
| Flux (ng/cm²/hour) | 0.70 | Not | Not | Not | Not | 3.94 | 1.33 | Not | Not | Not |
| (data from FIG. 8) | | tested | tested | tested | tested | | | tested | tested | tested |
| Cumulative amount | 51.3 ± 28.3 | 2.1 ± 0.5 | 39.7 ± | 65.3 ± | 17.6 ± | 87.3 ± 37.7 | 41.9 ± 17.0 | 67.3 ± 25.2 | 38.2 ± 19.2 | 1.9 ± 0.9 |
| at 24 hours (ng) | | | 14.2 | 29.9 | 7.9 | | | | | |
| (as illustrated in | | | | | | | | | | |
| FIG. 1) | | | | | | | | | | |
| Cumulative amount | 9.24 ± 2.25 | Not | Not | Not | Not | 32.92 ± 9.11 | 12.47 ± 3.67 | Not | Not | Not |
| at 24 hours (ng) | | tested | tested | tested | tested | | | tested | tested | tested |
| (as illustrated in | | | | | | | | | | |
| FIG. 8) | | | | | | | | | | |

Formulation numbers 1, 3, 4, 6 and 8 (solutions) were prepared by adding the respective solvents shown in Table 1 to a mixing vessel while stirring. The umeclidinium was subsequently added to the mixing vessel while stirring to give the desired solution formulations.

Formulation numbers 2, 5, 7, 9 and 10 (gels) were prepared by adding the respective solvents shown in Table 1 to a mixing vessel while stirring. The umeclidinium was subsequently added to the mixing vessel while stirring, followed by the addition of hydroxypropylcellulose to give the desired gel formulations.

The following additional formulations were also prepared to measure the viscosity of gel formulations according to the invention with varying levels of gelling agent (0, 1%, 1.5% and 2% of Klucel-MF):

TABLE 2

| | Formula No. | | | |
|---|---|---|---|---|
| | 11 | 12 | 13 | 14 |
| Component % w/w | % | % | % | % |
| Diethylene glycol | 25.00 | 25.00 | 25.00 | 25.00 |
| monoethyl ether | | | | |
| Propylene glycol | 56.40 | 56.40 | 56.40 | 56.40 |
| Benzyl alcohol | — | — | 1.00 | 1.00 |
| Purified water | 16.40 | 14.40 | 14.40 | 13.90 |
| Umeclidinium bromide | 2.195 | 2.195 | 2.195 | 2.195 |
| Hydroxypropyl- | — | 2.00 | 1.00 | 1.50 |
| cellulose (Klucel-MF) | | | | |
| TOTAL | 100 | 100 | 100 | 100 |
| Viscosity | — | 47,000 | 6,400 | 21,600 |
| (centipoises at 25° C.) | | | | |
| Flux | 3.49 | 1.45 | 2.14 | 1.93 |
| (ng/cm²/hour) | | | | |
| (data from FIG. 8) | | | | |
| Cumulative amount | 32.07 ± | 12.92 ± | 23.06 ± | 19.83 ± |
| at 24 hours (ng) (as | 8.16 | 3.10 | 6.55 | 5.98 |
| illustrated in FIG. 8) | | | | |

Formulation number 11 (solution) was prepared by adding the respective solvents shown in Table 2 to a mixing vessel while stirring. The umeclidinium was subsequently added to the mixing vessel while stirring to give the desired solution formulation.

Formulation numbers 12, 13 and 14 (gels) were prepared by adding the respective solvents shown in Table 2 to a mixing vessel while stirring. The umeclidinium was subsequently added to the mixing vessel while stirring, followed by the addition of hydroxypropylcellulose to give the desired gel formulations.

### Example 2

### In Vitro Skin Penetration Studies

The topical pharmaceutical compositions described in Example 1 were subjected to in vitro skin penetration studies to measure the skin flux. The following methodology was used:

### Methods and Material

Full thickness human skin is obtained from patients undergoing abdominoplasty at local hospitals. Immediately following collection, the skin is transferred to a plastic container with phosphate buffered saline (PBS) and kept at 4° C. during shipment. Upon arrival at the laboratory, the subcutaneous fat is removed from the skin samples. The full thickness skin is then placed on high-density foam blocks and dermatomed to a thickness of 500 μm using an Electro-Dermatome. The split thickness skin is then spread out on aluminium foil and placed in a water impermeable plastic bag. The air is removed, and the bag is heat sealed. The sample is stored at −80° C. until the time of the experiment. Previous experiments have shown that skin samples can be prepared and stored in this manner without damaging the rate limiting skin penetration physical barrier (stratum corneum).

### Method Summary

Diffusion cell type: Flow-through Channel diffusion cells
Skin membrane source: Human split-thickness from four donors dermatomed to a thickness of 500 μm
Skin replicates: at least 12 replicates per formulation and at least 3 different skin donors
Dosing of test articles: 10 μL/cm² (10 mg/cm²) approximately equivalent to 220 or 100 μg of API (salt)/skin section
Receiving fluid: Phosphate buffered saline (PBS) made fresh prior to each experiment, degassed under vacuum for 60 minutes, and placed in heat baths set to 37° C. to minimize air bubbles
Pump flow rate: 10.0 μL/min
Time points: Hourly collection, from 1 to 24 hours post-application

### Diffusion Cell Set-Up:

A flow-through diffusion cell system (Channel cells) developed in-house is used to assess drug delivery. The diffusion cells are placed in cell warming supports and heated using a circulating water bath set to 38° C. in order to maintain the skin surface temperature at 32±2° C. The cells are connected to multi-channel peristaltic pumps via Tygon tubing. The outlet of each diffusion cell is fitted with tubing angled to drip directly into square welled 96-well plates. Frozen split thickness skin is removed from the freezer and thawed on the bench for ˜30 minutes. Sections of skin are cut into 1×2 cm sections using razor blades and mounted onto the support rings in the diffusion cells, stratum corneum side up.

Donor compartment blocks are placed on the skin and secured using stainless steel springs to provide a leak proof seal, exposing a surface area of 1.0 cm². Air bubbles are removed by pulsing receiving fluid through cells at maximum flow rate. The pumps are adjusted to the pre-selected flow rate (10.0 μL/min). The diffusion systems are allowed to equilibrate for approximately 20 minutes. Cells showing leaks are replaced.

### Application of Test Articles:

Test articles are applied at a dose of 10 μL/cell (10 mg/cm²), which is spread uniformly onto the stratum corneum surface using a positive displacement pipette. Donor chambers are left open to ambient conditions.

### Receptor Fluid Sampling:

Receptor fluid is collected (preferably hourly) from 1 up to 24 hours post application into 96 well plates. Aliquots of the receiving fluid are transferred to 300 μL 96 well plates for high-throughput SPE-MS/MS or UPLC-MS/MS analysis. Prior to analysis, aliquots of acetonitrile or methanol containing internal standard are added to each well.

Sample Analysis: Samples are analyzed by high-throughput SPE-MS/MS, or MS or UPLC-MS/MS.

Data Analysis: Any diffusion cells with visible leakage are removed from analysis. Outliers are determined by the analyst and verified using JMP (SAS Institute Inc, v9.0).

Mean, standard deviation, and standard error of the mean are calculated using JMP. Student t-test is used to compare formulations. P-values of ≦0.05 are considered statistically significant.

The results of the studies are illustrated in Tables 1 and 2, and FIGS. 1 and 8.

### Example 3

### In Vitro Skin Penetration Studies (Umeclidinium Compared with Glycopyrrolate)

In vitro studies investigated skin distribution (epidermis/dermis) and the in vitro skin flux of the active ingredient delivered from (i) Formulation No. 1 comprising 2.2% umeclidinium bromide shown in Table 1 and (ii) a comparative formulation comprising 2% glycopyrrolate bromide.

At 6 hours, following a single finite topical dose of umeclidinium bromide or glycopyrrolate bromide on ex vivo human skin, the molar ratios of glycopyrrolate to umeclidinium (after correction for differences in dose) were a median (range) of 1.5 (0.4-5.8) in the epidermis and 1.2 (0.3-4.3) in the dermis. Thus, the amount of umeclidinium delivered to the dermis was the same order of magnitude (but slightly lower, on average), on a molar dose-normalized basis, as the amount of glycopyrrolate delivered to the dermis.

At 24 hours following a single finite topical dose in ex vivo human skin, the in-vitro skin flux for glycopyrrolate was a median (range) of 7.3 (1.0-65) fold higher than that of umeclidinium, on a molar basis after correction of dose.

Matrix-assisted laser desorption/ionization (MALDI) has emerged as a powerful and diverse technology for analyzing the spatial distribution of endogenous and exogenous compounds directly from a tissue section. [See Seeley E H, Caprioli R M. MALDI imaging mass spectrometry of human tissue: method challenges and clinical perspectives. Trends Biotechnol. 29(3), 136-143 (2011); and Svatos A. Mass spectrometric imaging of small molecules. Trends Biotechnol. 28(8), 425-434 (2010)]. MALDI has been shown to be a valuable tool in skin research with the potential to help with visualization of endogenous compounds as well as drug and its metabolites [See Enthaler B, Trusch M, Fischer M, Rapp C, Pruns J K, Vietzke J P., MALDI imaging in human skin tissue sections: focus on various matrices and enzymes. Anal Bioanal Chem. (2013); 405(4):1159-70]. MALDI imaging in human skin tissue sections: focus on various matrices and enzymes. MALDI was used to investigate the qualitative distribution of umeclidinium in the skin layers and to determine whether it penetrates into the dermis to the level of the sweat glands (2 mm or deeper). Following a single finite topical dose in ex vivo human skin, umeclidinium was shown to have a gradient within the skin, where the highest concentration was near the stratum corneum and this gradient migrated to the deeper layers of the skin over twenty-four hours. At twenty-four hours, umeclidinium penetrated to the deeper sections of the dermis and accumulated in the area where the sweat glands are present.

### Example 4

### Comparison of In Vivo Systemic Concentrations of Umeclidinium with Glycopyrrolate

The in vitro skin flux is not likely to be identical to the in vivo skin flux. That is, the in vivo skin flux is likely to be between 3 and 10 times, and possibly up to 100 times higher than the in vitro skin flux.

Simulations of human exposure following equimolar doses of umeclidinium and glycopyrrolate, using in vitro data from a single experiment and in vivo pharmacokinetic data after intra-venous dosing with umeclidinium or glycopyrrolate to healthy volunteers, showed that the concentrations of unbound (free) drug were lower for umeclidinium than for glycopyrrolate. Results from these simulations assuming worst case for umeclidinium (in vivo skin flux 100 times greater than the in vitro skin flux) and all potential cases for glycopyrrolate, showed that the probability that the unbound umeclidinium available systemically is greater than unbound glycopyrrolate available systemically is between <0.001% and 11% (Table 3). That is, it is likely that less umeclidinium is available systemically compared with glycopyrrolate. Accordingly, given that these compounds are equipotent at the muscarinic receptors, treatment with umeclidinium may potentially lead to fewer systemic adverse events compared with treatment with glycopyrrolate.

TABLE 3
Probability that systemic exposure of unbound umeclidinium
is greater than systemic exposure of unbound glycopyrrolate

| | | Probability that unbound, |
|---|---|---|
| | | dose-normalized* |
| | | Umeclidinium AUC > |
| Flux assumption | | unbound, dose-normal- |
| Umeclidinium | Glycopyrrolate | ized* Glycopyrrolate AUC |
| In-vivo = | In-vivo = 3 x in-vitro | 11.0% |
| 100 x | In-vivo = 10 x in-vitro | 2.22% |
| in-vitro | In-vivo = 100 x in-vitro | <0.001% |
| | | Probability that unbound, |
| | | dose-normalized* |
| | | Umeclidinium Cmax > |
| Flux assumption | | unbound, dose-normal- |
| Umeclidinium | Glycopyrrolate | ized Glycopyrrolate Cₘₐₓ |
| In-vivo = | In-vivo = 3 x in-vitro | 3.14% |
| 100 x | In-vivo = 10 x in-vitro | 0.46% |
| in-vitro | In-vivo = 100 x in-vitro | <0.001% |

*Dose normalized to 1% formulation

### Example 5

### Characterization of Systemic Exposure from Topical Administration of [¹⁴C] Umeclidinium to Axilla or Palm of Healthy Male Subjects

A clinical study has been conducted to characterize the pharmacokinetics, safety and tolerability of umeclidinium following a single radiolabelled dose administration of Formulation No. 1 to the axilla or the palm of healthy human subjects.

Diffusion of any compound through the skin is likely to be different between the axilla and the palm based on the known differences in the stratum corneum in these two areas of the body. The mean (SD) number of stratum corneum layers of the palm is 50, ranging from 30-70 layers. In contrast, the mean (SD) number of stratum corneum layers in the trunk is 13, ranging from 5-21 layers [Ya-Xian et al., Arch Dermatol Res 1999; 291: 555-559].

If one assumes that the in vivo flux through skin is up to 20-fold higher than the in vitro skin flux, Formulation no. 1 is likely to have a skin flux through axillary skin ranging from 0.33 to 127 ng/h/cm², and through palmar skin ranging from 0.04 to 49 ng/h/cm².

Previous safety studies showed that 0.5 mg (500 mcg) of inhaled umeclidinium administered once daily for 10 days was well tolerated in a thorough QT study. Therefore, a topical umeclidinium dose was aimed to produce a maximum exposure (based on Cₘₐₓ and AUC (0-∞)) with a low probability to exceed the systemic exposure levels (Cₘₐₓ and AUC(0-τ)) corresponding to 0.5 mg inhaled umeclidinium administered once daily for 10 days.

For this first dermal study, the dose administered was 165 mg of a solution comprising 2.2% w/w umeclidinium bromide (1.85% w/w umeclidinium cation) applied to a 40 cm² surface area. That is, the net amount of umeclidinium administered topically was 3.05 mg (i.e. 165 mg×1.85%). The systemic exposure (Cₘₐₓ and AUC(0-∞)) following administration of 3.05 mg of umeclidinium to the axilla was predicted not to exceed the exposure (Cₘₐₓ or AUC(0-tau)) resulting from 0.5 mg umeclidinium administered by inhalation once daily for 10 days.

In one embodiment, the doses to be administered topically for hyperhidrosis should range from a 0.01% to 5% by weight umeclidinium bromide solution applied to a maximum of 20% of the body surface area once daily, every second day, three times weekly, twice weekly or once weekly, as long as exposure is predicted to be equal to or lower than that from the inhaled umeclidinium clinical program. In another embodiment, the doses to be administered topically for hyperhidrosis should range from a 0.01% to 5% by weight umeclidinium bromide.

Dose calculations for the dermal study were based on several key parameters: (1) percentage of active pharmaceutical agent in the formulation (w/w), e.g. a solution comprising 2.2% w/w umeclidinium bromide has shown to have low irritation potential; (2) the steady state in vivo flux through skin (assuming absorption through the skin follows zero order kinetics similar to in vitro studies; 3) exposure time; 4) application surface area; 5) amount of product applied; and 6) predicted systemic concentration-time profiles relative to quantification limit and previous human exposures.

### Flux Through Axilla:

Due to the lipophilic nature (cLogP=6.96) of umeclidinium, the ability of the drug to partition into the receiving fluid in an in vitro assay may be limiting, as compared to in vivo conditions where the presence of plasma proteins and other solutes may act as an additional driving force to promote penetration. As such, the in vitro flux values are likely to be an underestimate. To estimate exposure following topical application in humans, simulations were conducted assuming a more realistic in vivo steady state flux that is 3- or 10-fold higher or, potentially, 100-fold higher than the in vitro skin flux.

### Flux Through Palm:

Given the differences in the number of layers of the stratum corneum in the palm relative to the axilla, and assuming that the flux difference between the palm and axilla is proportional to the difference in the number of stratum corneum layers, the steady state flux through palm was estimated to be on average 3.9-fold lower than the flux through axilla.

### Flux Changes Following Occlusion:

Occlusion is widely utilized in topical administration to increase the penetration of applied drugs. Systemic exposure under occlusive conditions was predicted assuming an increase in skin flux between 1.4-10 fold [Hafeez et al., Skin Pharmacol Physiol 2013; 26: 85-91].

### Exposure Time

To ensure that a sufficient amount of umeclidinium penetrates through the skin, the formulation was planned to be kept on the skin for 8 hours (equivalent to the average anticipated overnight application period for the formulation in the clinical setting).

### Application Surface Area

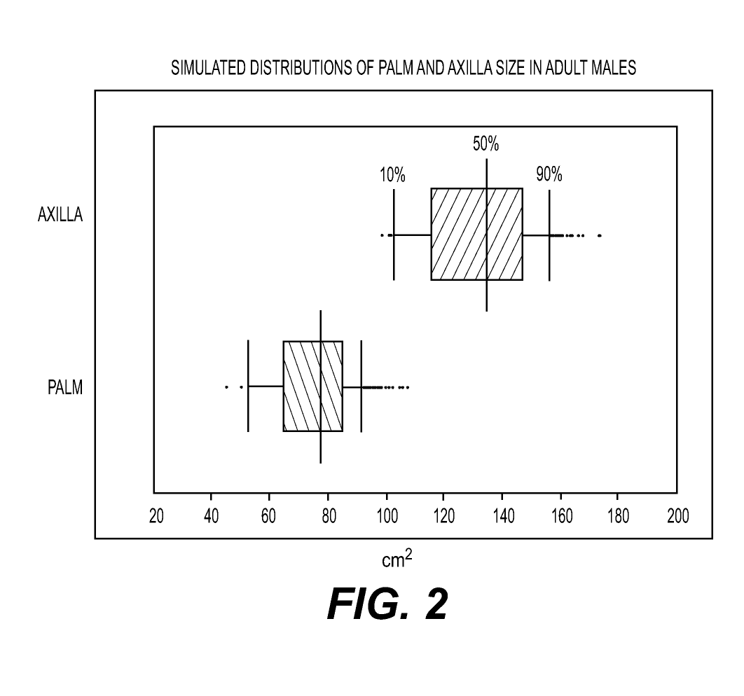

A one-size-fits-all template of 40 cm², designed to fit both the axilla and the palm of the male population, was used to mark the area of application on either site. FIG. 2 shows the distribution of palm and axilla size in adult males based on palm and axilla measurements [Agarwal P. Sahu S. Indian Journal of Plastic Surgery 2010; 43: 49-53 and Cowan-Ellsberry C et al., Regul Toxicol Pharmacol 2008; 52: 46-52]. Using ModelRisk, it was determined that the surface area that would fit both the axilla and the palm of 90% of the male population is 64.7 cm². In order to facilitate the attachment of a protective dome or of an occlusive dressing, a tolerance of approximately 1 cm was allowed on each of the sides of the template. Therefore, the final selected surface area of application was 40 cm² for this study, while the surface area for the clinical setting will be the entire axillas and/or palms.

### Amount of Formulation Applied

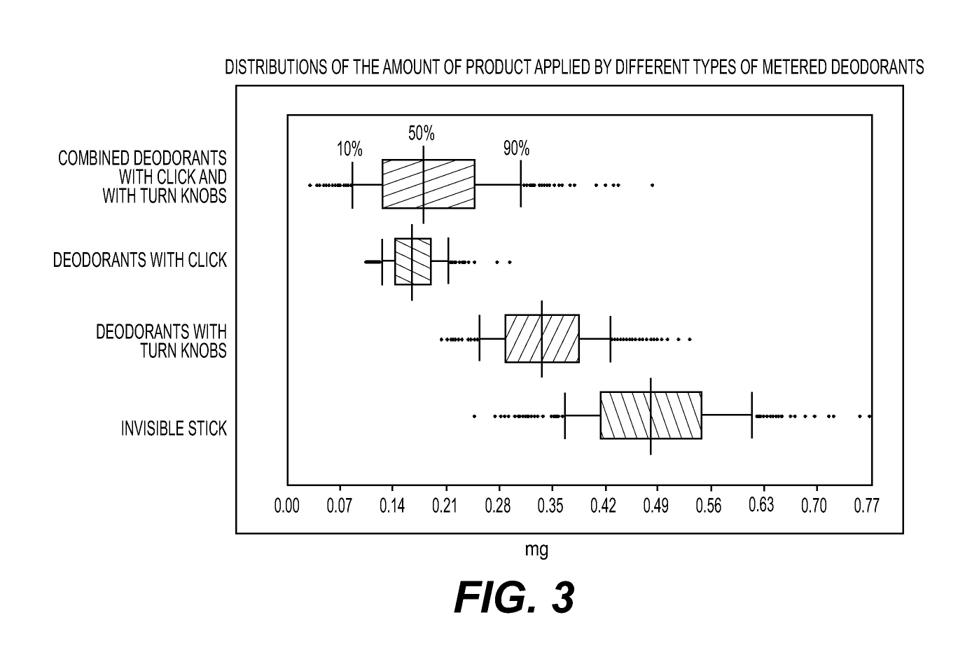

To determine the amount of formulation to be applied to the axilla or palm, an in-house experiment was conducted to evaluate three types of currently marketed deodorants with regard to the amount of formulation dispensed per actuation. FIG. 3 shows the individual and combined distributions of the amount of formulation applied using: 1) deodorants with click; 2) deodorants with turn knobs; and 3) invisible stick deodorants. The data presented in Table 4 illustrates that the mean amount of formulation dispensed by deodorants ranged between 165 to 538 mg per actuation. An amount of 165 mg of umeclidinium solution was considered sufficient to provide appropriate surface area coverage to 40 cm² without running

TABLE 4
Summary of the amount of formulation applied by one actuation
of different types of marketed controlled dose deodorants

| | Mean (90% CI) amount (mg) of |
|---|---|
| Deodorant type | formulation applied by one actuation |
| Click | 165 (115-229) |
| Turn knob | 336 (235-452) |
| Invisible stick | 538 (420-665) |
| Combined click and | 185 (71-344) |
| turn-knob deodorants | |

Based on key parameters 1) through 5) above, 165 mg of a 2.2% w/w umeclidinium bromide (1.85% w/w umeclidinium cation) solution was applied to a 40 cm² surface area on either the axilla or the palm (depending on the cohort into which the subject is enrolled). The calculated net amount of umeclidinium that was applied on the 40 cm² surface area is 3.05 mg.

### Predicted Concentrations Relative to Quantification Limit

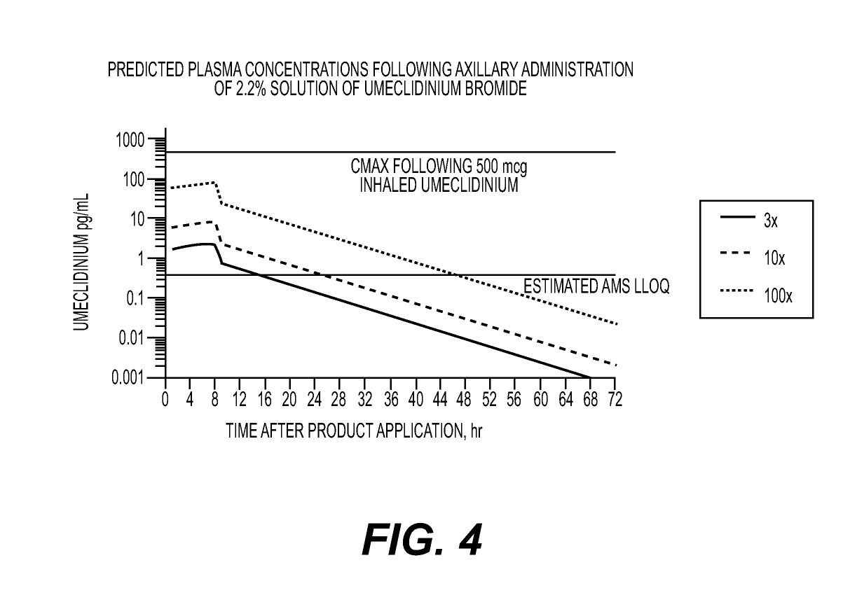

FIG. 4 shows the predicted pharmacokinetic profile of umeclidinium following dermal administration of 165 mg of the 2.2% w/w umeclidinium bromide (1.85% w/w umeclidinium cation) solution for 8 hours, on a surface area of 40 cm² to the axilla. These predictions were based on a population pharmacokinetic model from intravenous dosing and assumed that the skin absorption follows zero order kinetics with an in vivo steady state skin flux that was 3-, 10-, or 100-fold higher than the in vitro steady state skin flux.

Although simulations predict that concentrations will be above the estimated accelerated mass spectroscopy (AMS) lower limit of quantification (LLOQ) of ˜0.38 pg/mL for approximately 16-44 hours (not quite the 3-5× terminal phase half-life following inhaled dosing), it was expected to be acceptable to adequately characterize the area under the curve, the apparent half-life, the maximum concentrations, and the Tₘₐₓ of umeclidinium.

Umeclidinium has been well tolerated over the exposure (Cₘₐₓ and AUC(0-τ)) range observed following inhalation of up to 0.5 mg (which is a supra-therapeutic dose in QT study) once daily. Table 5 below presents the probabilities that the Cₘₐₓ and AUC (0-∞) following axillary administration exceed the Cₘₐₓ and AUC(0-τ) observed following inhaled supra-therapeutic 0.5 mg once daily doses. These results show that the probability of exceeding the Cₘₐₓ or AUC(0-τ) is less than 0.1% even when the in vivo flux is assumed to be 100-fold greater than the in vitro flux.

TABLE 5
Probability that Cₘₐₓ or AUC (0-∞) following topical administration
to the axilla of a solution comprising 2.2% w/w umeclidinium bromide
(1.85% w/w umeclidinium cation) exceeds the Cₘₐₓ or AUC(0-τ) observed
following 10 daily doses of 0.5 mg of umeclidinium administered
by inhalation

| | Probability that: | | | |
|---|---|---|---|---|
| | | Median | | Median |
| | Cₘₐₓ | Cₘₐₓ | AUC(0-∞) | AUC(0-∞) |
| | (Dermal) > | (Dermal) > | (dermal) > | (dermal) > |
| Assumption that | Cₘₐₓ | Cₘₐₓ | AUC(0-τ) | AUC(0-τ) |
| in-vivo flux = | (Inhaled) | (Inhaled) | (inhaled) | (inhaled) |
| 3 x in-vitro flux | <0.1% | | | |
| 10 x in-vitro flux | | | | |
| 100 x in-vitro flux | <0.1% | <0.1% | <0.1% | 5.9% |

Once Cohorts A and B are completed, new simulations will be conducted to estimate the exposure following palmar administration (occluded and unoccluded), assuming that the difference in exposure between palm and axilla is proportional to the difference in the number of stratum corneum layers. When data are available from all 4 cohorts, the in vivo skin flux will be calculated for axillary and palmar administration with and without occlusion, if possible.

### Study Design

A single-center, single dose, open-label study to characterize the pharmacokinetics, safety and tolerability of topically applied umeclidinium bromide solution following administration to the axilla or palm of healthy male subjects is being undertaken, with three cohorts (A, B and D) completed to date. Up to four cohorts were to be enrolled (Cohort A: unoccluded axilla; Cohort B: occluded axilla; Cohort C: unoccluded palm; Cohort D: occluded palm). Six subjects were to be dosed in each cohort and complete the study procedures.

On the day of dosing, subjects will receive the study drug applied to the axilla or palm. Each subject will only receive a single dose of study drug which will be applied to the test site and remain on the application site for 8 hours. Serial blood samples will be collected throughout treatment. An interim analysis using simulations will be conducted after each cohort to determine the likelihood of exceeding the safety cover for Cₘₐₓ or AUC and the likelihood of obtaining quantifiable concentrations sufficient to determine the half-life in the subsequent cohorts. Because simulations based on data from Cohort A or Cohort B suggested that concentrations following application to the unoccluded palm will be non-quantifiable, the dose was applied only to Cohort D (occluded palm), and Cohort C (unoccluded palm) will not be conducted.

Preliminary Results from Cohorts A, B and C

Following administration to unoccluded axilla (Cohort A), the mean (range) percent dose recovered from the skin surface at the end of the 8 hour application period was 80.6% (77.7-84.1%). The majority of the subjects had non-quantifiable plasma concentrations or incomplete profiles. One subject had quantifiable plasma concentrations up to 72 hours post-dose, followed by non-quantifiable plasma concentrations at the follow-up visit i.e., 10-14 days post-dose. Since umeclidinium is known to have a flip-flop pharmacokinetics (i.e., absorption-rate related pharmacokinetics), these results indicate that umeclidinium is still being absorbed at least 72 hours after administration of a single dose, but not at 10 to 14 days (e.g., absorption between 3 and 10 days is unknown). The median time to maximum plasma concentration (Cₘₐₓ) was 13 hours (range: 12 h-30 h). The mean (CV %) Cₘₐₓ was 2.33 pg/mL (144%). Due to the large amount of plasma concentrations below the limit of quantitation (BLQ) in the distribution and elimination phase, calculation of terminal elimination half-life (t_{1/2}) and area under the plasma concentration-time curve from zero to infinity(AUC(0-∞) was not possible in the majority of the subjects.

Following administration to occluded axilla (Cohort B), the mean (range) percent dose recovered from the skin surface was 75.4% (69.2%-81.7%). The samples were quantifiable up to 16-72 hours post dose, with adequate profiles in 5 of 6 subjects. The median time to maximum concentration was 13 hours (range: 2 h-24 h). The mean (CV %) Cₘₐₓ was 7.92 mg/mL (122%). The ratio of Cₘₐₓ in occluded axilla to unoccluded axilla averaged 9.1 fold (range 2- to 33.6-fold). Due to highly variable or BLQ concentrations in the terminal elimination phase, calculation of the t_{1/2} and AUC (0-∞) was possible in only two subjects. As with the unoccluded axilla, the plasma concentration-time profiles of some subjects suggest that the drug is still being absorbed by 72 hours but not at 10 to 14 days post-treatment. Thus, dosing once daily may not be necessary and dosing as infrequently as once weekly may be possible, depending on future simulations.

Following administration to occluded palm (Cohort D), the mean (range) percent dose recovered from the skin surface was 52.5% (45.6%-59.9%). There were no quantifiable plasma concentrations.

### Population Pharmacokinetic Model

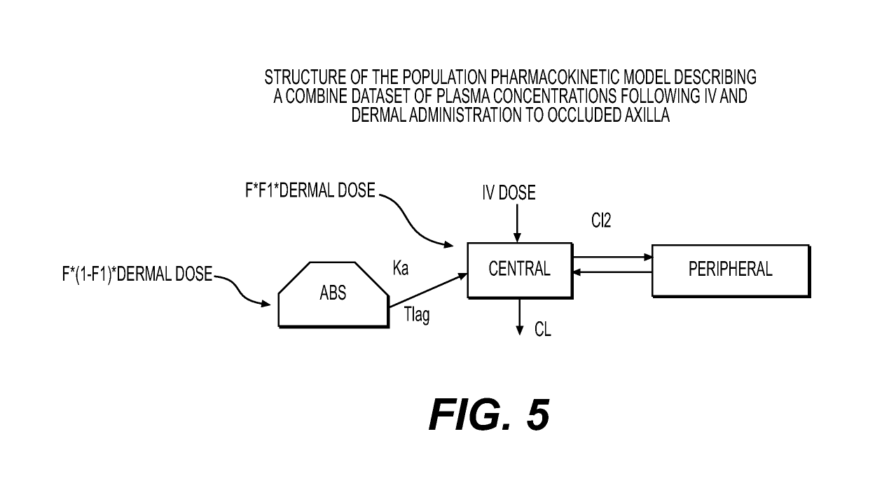

Umeclidinium concentration-time data following dermal administration to the occluded axilla was combined with concentration-time data following a 30 minute IV infusion of 65 mcg. The combined pharmacokinetic dataset was best described by a two compartment population pharmacokinetic model (PopPK), with first order elimination from the central compartment, and two sequential absorption processes: a zero order process followed by a first order process with lag time (FIG. 5). The estimated mean (relative standard error [RSE %]) PopPK model parameters are shown in Table 6. The estimated absolute plasma bioavailability following administration to occluded axilla was 0.19% (55.3%). Of the drug that was available systemically, 9.70% (66.7%) was absorbed through a zero order process.

TABLE 6
Parameter values of the population pharmacokinetic
model describing the combined plasma data following
IV and dermal administration to occluded axilla.

| | | | Inter-individual |
|---|---|---|---|
| Parameter | | Mean (RSE %) | variability |
| V1 | Volume of central | 7726 (17.6%) | 45.4% |
| (mL) | compartment | | |
| CL | Elimination | 45241 (23.6%) | 100% |
| (mL/h) | clearance | | |
| V2 | Volume of | 261058 (57.3%) | |
| (mL) | peripheral | | |
| | compartment | | |
| CL2 | Intercompartmental | 39376 (26.0%) | 92.5% |
| (mL/h) | Clearance | | |
| Ka | First order | 0.094 (54.1%) | 94.9% |
| | absorption rate | | |
| | constant | | |
| F1 | Fraction of the | 0.097 (66.7%) | |
| | bioavailable drug | | |
| | absorbed through a | | |
| | zero order process. | | |
| F | Absolute plasma | 0.0019 (55.3%) | |
| | bioavailability | | |
| | following | | |
| | administration to | | |
| | occluded axilla | | |
| Tlag | Lag time for the first | 8.61 (15.3%) | 169% |
| (hr) | order absorption | | |
| | process | | |

Zero order rate estimate = F1*F*Dose/Tlag = 1.6 ng/h/cm², range: 0.05-45.9 ng/h/cm²
Residual error 34.4%
AIC = 1274
RSE % = the relative standard error calculated as standard error/final parameter estimate *100. AIC = Akaike information criterion.

### Predicted Plasma Concentrations Following Repeat Dose Administration

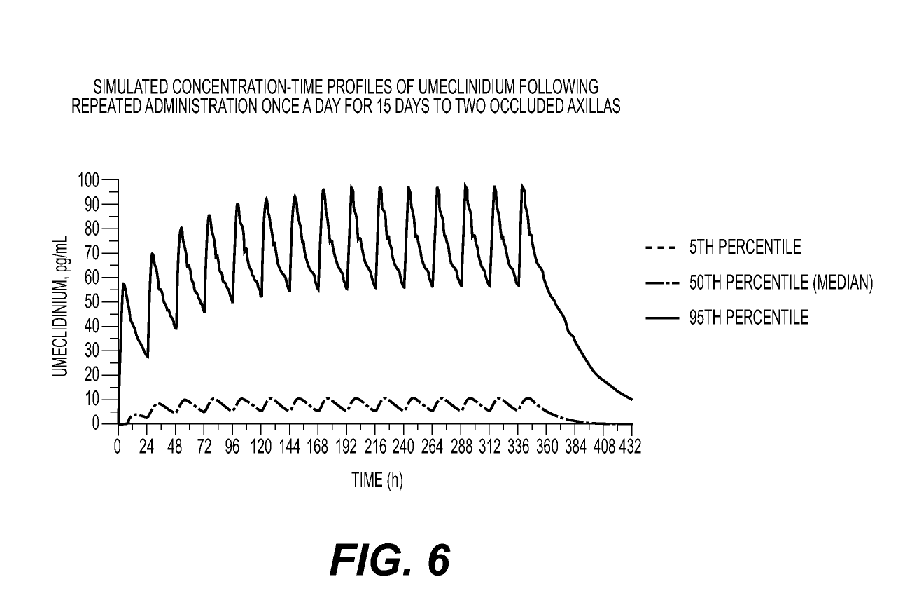

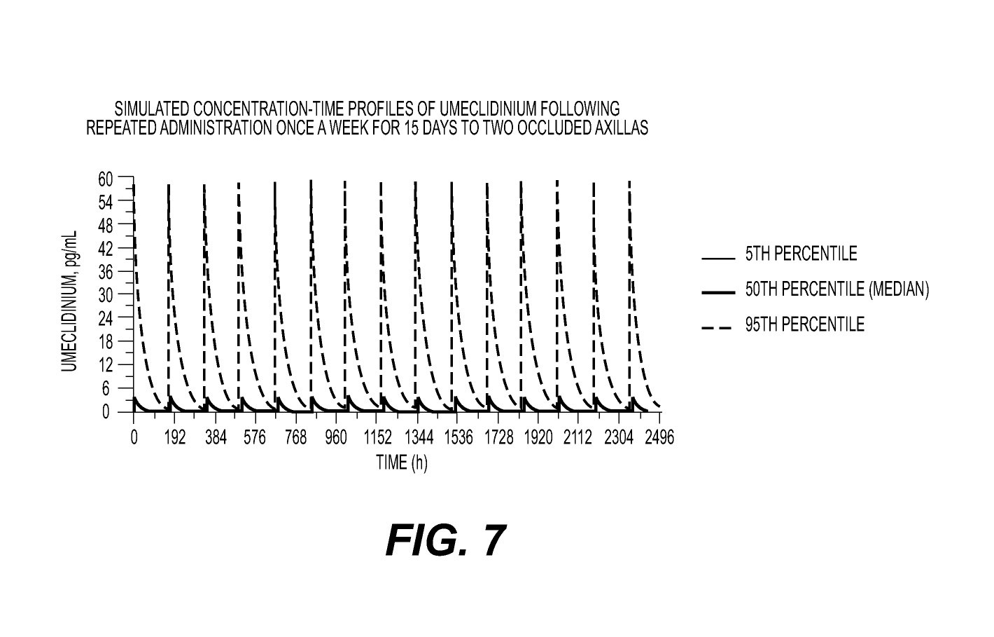

The PopPK model was used to simulate plasma concentration-time profiles following repeated once daily doses (QD) to occluded axilla for 15 days (FIG. 6) or repeated once weekly doses to occluded axilla for 15 weeks (FIG. 7). Simulations were performed assuming 1) 170 mg (±20% CV) of the 1.85% (w/w) formulation were applied to each of the axillas; 2) formulation was applied to both males and females; 3) male and female axilla sizes were based on axilla measurements as described in Cowan-Ellsberry C et al., Regul Toxicol Pharmacol 2008; 52: 46-52. Data in Table 6 represent Day 15 pharmacokinetic parameter determined by noncompartmental analysis (NCA) of the simulated plasma concentration-time profiles following 15 QD doses to occluded axilla.

TABLE 7
Day 15 pharmacokinetic parameters determined by NCA analysis
of the simulated plasma concentration-time profiles following 15 QD
doses to occluded axilla^{a}. PK parameters were calculated assuming
a lower limit of quantitation of 0.348 pg/mL.

| Parameter name (Unit) | Value |
|---|---|
| Accumulation Ratio | 1.28 (1.00-3.41) |
| Cmax (pg/mL) | 30.5 (146) [2.49-396] |
| AUC24 (h*pg/mL) | 251 (153) [20.7-3970] |
| AUCinf (h*pg/mL) | 396 (226) [22.4-16873] |
| t_{1/2} (h) | 11 (3.30-48.0) |

^{a}Data represent geometric mean values (geometric coefficient of variation [in percent]) [range] for all parameters except the accumulation ratio and half-life, which are represented as median (90% confidence interval). AUCtau, area under the plasma concentration time profile from predose to 24 h; AUCinf, area under the plasma concentration time profile from predose to infinity; Cmax, maximum concentration; t_{1/2}, half-life

Data in Table 8 represent pharmacokinetic parameters after last dose following 15 weekly doses to occluded axilla, determined by non-compartmental analysis of the simulated plasma concentration-time profiles.

TABLE 8
Pharmacokinetic parameters (last dosing day) determined by NCA
analysis of the simulated plasma concentration-time profiles following
15 weekly doses to occluded axilla^{a}. Parameters were calculated
assuming a lower limit of quantitation of 0.348 pg/mL.

| Parameter name (Unit) | Value |
|---|---|
| Accumulation Ratio | 1.00 (1.00-1.09) |
| Cmax (pg/mL) | 25 (143) [2.24-349] |
| AUC24 (h*pg/mL) | 306 (163) [17.3-227] |
| AUCinf (h*pg/mL) | 329 (181) [21.0-3940] |
| t_{1/2} (h) | 10.5 (3.07-45.6) |

^{a}Data represent geometric mean values (geometric coefficient of variation [in percent]) [range] for all parameters except the accumulation ratio and half-life, which are represented as median (90% confidence interval). AUCtau, area under the plasma concentration time profile from predose to 24 h; AUCinf, area under the plasma concentration time profile from predose to infinity; Cmax, maximum concentration; t_{1/2}, half-life.

The probability that the exposure following daily or weekly doses of umeclidinium axillary administration to exceed the Cₘₐₓ and AUC(0-τ) observed following inhaled supra-therapeutic 0.5 mg once daily doses is less than 2.4%.

### Predicted Receptor Occupancy in Dermis Following Dermal Administration at Steady State.

A semi-mechanistic simulation model is built to estimate % receptor occupancy in the dermis. One method is to use a simulation model consisting of a “dermis” compartment, a systemic central and a peripheral compartment with first order elimination from the central compartment, and a zero order absorption rate from the skin surface to the dermis. The diffusion rate within dermis to capillaries is calculated based on the rate of diffusion of small molecules in tissues (0.162 cm/h), and the average distance to capillaries assuming it to be about 37.5 μm. Receptor occupancy is modelled with receptor-drug on/off rates, and a ratio determined by Ki (0.062 nM).

Simulation results would predict a % receptor occupancy in skin at steady state of 20-100%.

All publications, including but not limited to patents and patent applications, cited in this specification are herein incorporated by reference as if each individual publication were specifically and individually indicated to be incorporated by reference herein as though fully set forth.

The above description fully discloses the present disclosure including preferred embodiments thereof. Modifications and improvements of the embodiments specifically disclosed herein are within the scope of the following claims. Without further elaboration, it is believed that one skilled in the art can, using the preceding description, utilize the present disclosure to its fullest extent. Therefore, the Examples herein are to be construed as merely illustrative and not a limitation of the scope of the present disclosure in any way. The embodiments of the present disclosure in which an exclusive property or privilege is claimed are defined as follows.

What is claimed is:

1. A method for the treatment of and/or the prophylaxis of excessive sweating or hyperhidrosis in a human in need thereof, comprising applying to the skin of said human a therapeutically effective amount of a composition comprising 4-[hydroxy(diphenyl)methyl]-1-{2-[(phenylmethyl)oxy]ethyl}-1-azoniabicyclo[2.2.2]octane, and a pharmaceutically acceptable anion thereof (umeclidinium), and a pharmaceutically acceptable carrier thereof.

2. The method according to claim 1, wherein the pharmaceutically acceptable anion is selected from chloride, bromide, iodide, hydroxide, sulfate, nitrate, phosphate, acetate, trifluoroacetate, fumarate, citrate, tartrate, oxalate, succinate, mandelate, methanesulfonate or p-toluenesulfonate.

3. The method according to claim 2, wherein the pharmaceutically acceptable anion is bromide.

4. The method according to claim 1, wherein the 4-[hydroxy(diphenyl)methyl]-1-{2-[(phenylmethyl)oxy]ethyl}-1-azoniabicyclo[2.2.2]octane and a pharmaceutically acceptable anion thereof is present in an amount from about 0.1% to about 10% by weight applied to a maximum of 20% of the BSA.

5. The method according to claim 4, wherein the umelcidinium is present in an amount with a dose of 0.01 to 10,000 mg.

6. The method according to claim 4, wherein the individual dose applied to the affected area is from about 0.37 to about 31.6 mg/dose of 4-[hydroxy(diphenyl)methyl]-1-{2-[(phenylmethyl)oxy]ethyl}-1-azoniabicyclo[2.2.2]octane and a pharmaceutically acceptable anion thereof (equivalent to 0.44 to 38 mg/dose umeclidinium bromide).

7. The method according to claim 1, wherein the umeclidinium produces a maximum systemic plasma level of less than 1607 pcg/mL at steady state.

8. The method according to claim 1, wherein the umeclidinium produces a systemic AUC(0-tau) at steady state of less than 2541 hr*pcg/mL.

9. The method according to claim 1, wherein the umeclidinium produces a maximum systemic plasma level of less than 1607 pcg/mL at steady state and a systemic AUC(0-tau) at steady state of less than 2541 hr*pcg/mL.

10. The method according to claim 1, wherein the composition is formulated for topical administration directly to an affected region of the human patient.

11. The method according to claim 10, wherein the composition is formulated in unit dose form.

12. The method according to claim 10, wherein the medicament is in the form of a solution, a gel, a cream, an ointment, a lotion, a spray, an aerosol spray or an aerosol foam.

13. The method according to claim 1, wherein the composition is applied to the affected area twice daily, once daily, once every second day, three times weekly, twice weekly or once weekly.

14. A pharmaceutical composition for topical administration comprising a therapeutically effective amount of umeclidinium, and at least one pharmaceutically acceptable solvent.

15. The pharmaceutical composition according to claim 14, wherein the composition has a skin flux of at least 0.2 ng/cm²/hour measured in vitro using ex vivo human abdominal skin.

16. The pharmaceutical composition according to claim 14, wherein the solvent comprises a mixture of water and at least one water miscible organic solvent.

17. The pharmaceutical composition according to claim 16, wherein the water is present in an amount from about 5% to about 55% by weight and the water miscible organic solvent is present in an amount from about 45% to about 90% by weight, based on the total weight of the composition.

18. The pharmaceutical composition according to claim 16, wherein the water miscible organic solvent is an alcohol selected from the group consisting of methyl alcohol, ethyl alcohol, isopropyl alcohol, n-propyl alcohol, isobutyl alcohol, n-butyl alcohol, t-butyl alcohol, benzyl alcohol, tetrahydrofurfuryl alcohol, butylene glycol, diethylene glycol, diethylene glycol monoethyl ether, dipropylene glycol, ethylene glycol, ethyl hexanediol, ethylene glycol, 1,2-hexanediol, hexylene glycol, pentylene glycol, propanediol and propylene glycol, and mixtures thereof.

19. The pharmaceutical composition according to claim 16, wherein the water miscible organic solvent comprises a mixture of diethylene glycol monoethyl ether and propylene glycol.

20. The pharmaceutical composition according to claim 16 further comprising a penetration enhancer.

21. The topical pharmaceutical composition according to claim 14, wherein the composition is a solution.

22. The topical pharmaceutical composition according to claim 14, wherein the composition is a gel.

23. A topical pharmaceutical composition comprising:
umeclidinium present in an amount from about 0.5% to about 5% by weight;
water in an amount from about 5% to about 55% by weight; and
at least one water miscible organic solvent in an amount from about 45% to about 90% by weight, and
wherein all % are based on the total weight of the composition.

24. A topical pharmaceutical composition comprising:
umeclidinium present in an amount from about 0.5% to about 5% by weight;
water in an amount from about 5% to about 25% by weight; and
at least one water miscible organic solvent in an amount from about 70% to about 90% by weight, which is a mixture comprising diethylene glycol monoethyl ether and propylene glycol, and
wherein all % are based on the total weight of the composition.

25. A method of reducing or preventing perspiration comprising applying to the skin of a human a therapeutically effective amount of a composition of 4-[hydroxy(diphenyl)methyl]-1-{2-[(phenylmethyl)oxy]ethyl}-1-azoniabicyclo[2.2.2]octane, and a pharmaceutically acceptable anion thereof (umeclidinium), and a pharmaceutically acceptable carrier thereof.

26. A method of treating or minimizing, or preventing undesirable odors associated with human sweat glands and skin in a human in need thereof, comprising applying to the skin of said human a therapeutically effective amount of a 4-[hydroxy(diphenyl)methyl]-1-{2-[(phenylmethyl)oxy]ethyl}-1-azoniabicyclo[2.2.2]octane, and a pharmaceutically acceptable anion thereof (umeclidinium), and a pharmaceutically acceptable carrier thereof.

27. A method for the prophylaxis or treatment of excessive sweating or hyperhidrosis comprising administering either sequentially or simultaneously, to a patient in need thereof, a pharmaceutical product comprising a therapeutically effective amount of a first therapeutic agent which is 4-[hydroxy(diphenyl)methyl]-1-{2-[(phenylmethyl)oxy]ethyl}-1-azoniabicyclo[2.2.2]octane and a pharmaceutically acceptable anion thereof (umeclidinium), and at least one other therapeutic agent.

28. A pharmaceutical composition for topical administration comprising a therapeutically effective amount of umeclidinium, and at least one pharmaceutically acceptable solvent, wherein the composition produces a maximum plasma level of umeclidinium at steady state of less than 1607 pcg/mL in a human upon administration to the skin.

29. A pharmaceutical composition for topical administration comprising a therapeutically effective amount of umeclidinium, and a pharmaceutically acceptable solvent, wherein the composition produces an AUC(0-tau) at steady state of less than 2541 hr*pcg/mL, in a human upon administration to the skin.

30. A pharmaceutical composition for topical administration comprising a therapeutically effective amount of umeclidinium, and a pharmaceutically acceptable solvent, wherein the composition produces a maximum plasma level of umeclidinium less than 1607 pcg/mL, and an AUC(0-tau) at steady state of less than 2541 hr*pcg/mL, in a human upon administration to the skin.
